(19) United States
(12) Patent Application Publication
(10) Pub. No.: US 2022/0370600 A1
(43) Pub. Date: Nov. 24, 2022

(54) MULTIGENIC MVA-SARS-COV-2 VACCINE

(71) Applicant: BOARD OF REGENTS, THE UNIVERSITY OF TEXAS SYSTEM, Austin, TX (US)

(72) Inventors: Haitao HU, Galveston, TX (US); Chaojie Zhong, Galveston, TX (US)

(21) Appl. No.: 17/747,358

(22) Filed: May 18, 2022

Related U.S. Application Data
(60) Provisional application No. 63/191,824, filed on May 21, 2021.

(51) Int. Cl. A61K 39/215; A61P 37/04; A61K 39/39

(52) U.S. Cl. CPC A61K 39/215; A61P 37/04; A61K 39/39; A61K 2039/55561

(57) ABSTRACT

The present invention includes compositions and methods of making and using an immunogenic protein for mucosal delivery comprising at least 90%, 91%, 92%, 93%, 94%, 95%, 96%, 97%, 98%, 99%, or 100% amino acid identity to a multigenic coronavirus vaccine on a modified vaccinia ankara (MVA) vector that expresses a viral nucleoprotein (N) protein and a spike (S) protein.

### CROSS-REFERENCE TO RELATED APPLICATIONS

This patent application claims priority to U.S. Provisional Patent Application No. 63/191,824 filed on May 21, 2021 and entitled “Multigenic MVA-SARS-COV-2 Vaccine”, the contents of which are hereby incorporated by reference in their entirety.

### TECHNICAL FIELD OF THE INVENTION

The present invention relates in general to the field of vaccines, and more particularly, to a novel multigenic MVA-SARS-CoV-2 vaccine for protection against SARS CoV-2.

### STATEMENT OF FEDERALLY FUNDED RESEARCH

None.

### INCORPORATION-BY-REFERENCE OF MATERIALS FILED ON COMPACT DISC

The present application includes a Sequence Listing which has been submitted in ASCII format via EFS-Web and is hereby incorporated by reference in its entirety. Said ASCII copy, created on May 18, 2022, is named UTMB1068_ST25.txt and is 36,864 bytes in size.

### BACKGROUND OF THE INVENTION

Without limiting the scope of the invention, its background is described in connection with coronavirus infection.

Since its detection in December 2019, COVID-19 has rapidly spread [1] and caused a major pandemic with more than 110 million human infections and 2.6 million deaths worldwide. Safe and protective vaccines are critical to mitigate virus infection and spread of the disease [2-4]. To date, a large number of SARS-CoV-2 vaccine candidates based on various platforms are in development, including DNA [5, 6], mRNA [7-9], viral vectors [10-18], protein subunit [19-21], and inactivated vaccine [22]. To date, some of these vaccines have demonstrated promising preclinical or clinical efficacy, among which the two mRNA vaccines and the Ad26 viral vector vaccine were approved by the FDA for use in the U.S. under Emergency Use Authorization [23].

Despite these advances, a need remains for novel compositions and methods for providing enhanced immunization of subjects to coronavirus and other viral infections.

### SUMMARY OF THE INVENTION

In one embodiment, the present invention includes an immunogenic protein for mucosal delivery comprising, consisting essentially of, or consisting of: at least 90%, 91%, 92%, 93%, 94%, 95%, 96%, 97%, 98%, 99%, or 100% amino acid identity to a multigenic coronavirus vaccine on a modified vaccinia ankara (MVA) vector that expresses a viral nucleoprotein (N) protein and a spike (S) protein. In one aspect, the immunogenic protein is formulated into an intranasal, pulmonary, or alveolar immunization. In another aspect, the immunogenic protein are SEQ ID NOS:1 and 3. In another aspect, the immunogenic proteins are encoded by SEQ ID NOS: 2 and 4. In another aspect, an intranasal immunization induces at least one of: local T-cell responses in the lung; granzyme B-expressing cytotoxic CD8⁺ T cells; reduced viral loads; or reduced lung inflammation. In another aspect, the immunogenic protein is weakly or non-antigenic when administered intramuscularly. In another aspect, the immunogenic protein is effective without neutralizing antibodies. In another aspect, the immunogenic protein is included in a composition that further comprises an adjuvant selected from at least one of alum, aluminum hydroxide, aluminum phosphate, calcium phosphate hydroxide, cytosine-guanosine oligonucleotide (CpG-ODN) sequence, granulocyte macrophage colony stimulating factor (GM-CSF), monophosphoryl lipid A (MPL), poly(I:C), MF59, Quil A, N-acetyl muramyl-L-alanyl-D-isoglutamine (MDP), FIA, montanide, poly (DL-lactide-coglycolide), squalene, glucopyranosyl lipid adjuvant (GLA), GLA-Alum, 3M-052, a glucopyranosyl lipid adjuvant GLA emulsion with squalene (GLA-SE), virosome, AS03, ASO4, IL-1, IL-2, IL-3, IL-4, IL-5, IL-6, IL-7, IL-8, IL-10, IL-12, IL-15, IL-17, IL-18, STING, CD40L, pathogen-associated molecular patterns (PAMPs), damage-associated molecular pattern molecules (DAMPs), Freund's complete adjuvant, Freund's incomplete adjuvant, transforming growth factor (TGF)-beta antibody or antagonists, A2aR antagonists, lipopolysaccharides (LPS), Fas ligand, Trail, lymphotactin, Mannan (M-FP), APG-2, Hsp70 and Hsp90, pattern recognition receptor ligands, TLR3 ligands, TLR4 ligands, TLR5 ligands, TLR7/8 ligands, or TLR9 ligands. In another aspect, the coronavirus is MERS, SARS, SARS-CoV-2, or variants thereof.

In another embodiment, the present invention includes a method of stimulating an immune response in an animal comprising, consisting essentially of, or consisting of: administering to the animal a composition comprising at least 90%, 91%, 92%, 93%, 94%, 95%, 96%, 97%, 98%, 99%, or 100% amino acid identity to a multigenic coronavirus vaccine on a modified vaccinia ankara (MVA) vector that expresses viral nucleoprotein (N) and S protein. In one aspect, the immunogenic proteins is formulated into an intranasal, pulmonary, or alveolar immunization. In another aspect, the antigenic proteins are SEQ ID NOS:1 and 3. In another aspect, the immunogenic proteins are encoded by SEQ ID NOS: 2 and 4. In another aspect, an intranasal immunization induces at least one of: local T-cell responses in the lung; granzyme B-expressing cytotoxic CD8⁺ T cells; reduced viral loads; or reduced lung inflammation. In another aspect, the immunogenic protein is weakly or non-antigenic when administered intramuscularly. In another aspect, the immunogenic protein is effective without neutralizing antibodies. In another aspect, the method further comprises adding an adjuvant selected from at least one of alum, aluminum hydroxide, aluminum phosphate, calcium phosphate hydroxide, cytosine-guanosine oligonucleotide (CpG-ODN) sequence, granulocyte macrophage colony stimulating factor (GM-CSF), monophosphoryl lipid A (MPL), poly(I:C), MF59, Quil A, N-acetyl muramyl-L-alanyl-D-isoglutamine (MDP), FIA, montanide, poly (DL-lactide-coglycolide), squalene, glucopyranosyl lipid adjuvant (GLA), GLA-Alum, 3M-052, a glucopyranosyl lipid adjuvant GLA emulsion with squalene (GLA-SE), virosome, AS03, ASO4, IL-1, IL-2, IL-3, IL-4, IL-5, IL-6, IL-7, IL-8, IL-10, IL-12, IL-15, IL-17, IL-18, STING, CD40L, pathogen-associated molecular patterns (PAMPs), damage-associated molecular pattern molecules (DAMPs), Freund's complete adjuvant, Freund's incomplete adjuvant, transforming growth factor (TGF)-beta antibody or antagonists, A2aR antagonists, lipopolysaccharides (LPS), Fas ligand, Trail, lymphotactin, Mannan (M-FP), APG-2, Hsp70 and Hsp90, pattern recognition receptor ligands, TLR3 ligands, TLR4 ligands, TLR5 ligands, TLR7/8 ligands, or TLR9 ligands. In another aspect, the coronavirus is MERS, SARS, SARS-CoV-2, or variants thereof.

In another embodiment, the present invention includes a method for production of an immunogenic protein comprising, consisting essentially of, or consisting of: (a) providing a cell containing an expression cassette having a nucleic acid encoding an immunogenic protein that has at least 90%, 91%, 92%, 93%, 94%, 95%, 96%, 97%, 98%, 99%, or 100% amino acid identity to a multigenic SARS-CoV-2 vaccine on a modified vaccinia ankara (MVA) vector that expresses viral nucleoprotein (N) and S protein; and (b) growing the virus in a cell under conditions in which the nucleic acid is expressed and the immunogenic protein is produced. In one aspect, the method further comprises the step of recovering the immunogenic protein. In another aspect, th a promoter is selected from the group consisting of constitutive promoters and tissue specific promoters. In another aspect, the immunogenic proteins are formulated into an immunization. In another aspect, the immunogenic protein is formulated into an intranasal, pulmonary, or alveolar immunization. In another aspect, the antigenic proteins are SEQ ID NOS:1 and 3. In another aspect, the immunogenic proteins are encoded by SEQ ID NOS: 2 and 4. In another aspect, the intranasal immunization induces at least one of: local T-cell responses in the lung; granzyme B-expressing cytotoxic CD8⁺ T cells; reduced viral loads; or reduced lung inflammation. In another aspect, the immunogenic protein is weakly or non-antigenic when administered intramuscularly. In another aspect, the immunogenic protein is effective without neutralizing antibodies. In another aspect, the coronavirus is MERS, SARS, SARS-CoV-2, or variants thereof.

In another embodiment, the present invention includes a nucleic acid encoding a protein comprising, consisting essentially of, or consisting of: an immunogenic protein that has at least 90%, 91%, 92%, 93%, 94%, 95%, 96%, 97%, 98%, 99%, or 100% amino acid identity to a multigenic coronavirus vaccine on a modified vaccinia ankara (MVA) vector that expresses viral nucleoprotein (N) and S protein. In one aspect, the antigenic proteins are SEQ ID NOS:1 and 3. In another aspect, the immunogenic proteins are encoded by SEQ ID NOS: 2 and 4. In another aspect, the coronavirus is MERS, SARS, or SARS-CoV-2, or variants thereof.

In another embodiment, the present invention includes a vector that comprises, consisting essentially of, or consisting of: a nucleic acid that encodes an immunogenic protein that has at least 90%, 91%, 92%, 93%, 94%, 95%, 96%, 97%, 98%, 99%, or 100% amino acid identity to a multigenic coronavirus vaccine on a modified vaccinia ankara (MVA) vector that expresses a viral nucleoprotein (N) protein and a spike (S) protein.

In another embodiment, the present invention includes a host cell that comprises, consisting essentially of, or consisting of: a vector that expresses an immunogenic protein that has at least 90%, 91%, 92%, 93%, 94%, 95%, 96%, 97%, 98%, 99%, or 100% amino acid identity to a multigenic coronavirus vaccine on a modified vaccinia ankara (MVA) vector that expresses a viral nucleoprotein (N) protein and a spike (S) protein.

### BRIEF DESCRIPTION OF THE DRAWINGS

For a more complete understanding of the features and advantages of the present invention, reference is now made to the detailed description of the invention along with the accompanying figure(s) and in which:

FIGS. 1A-1E. Vaccine design, generation and characterization. (FIG. 1A) Recombinant MVA vaccine cloning. cDNA sequence containing SARS-CoV2 full-length SARS-CoV2 S or N was respectively cloned into MVA transfer plasmid pLW17 and pLW9. The S and N insert sequences were respectively engineered to contain mNeonGreen and mScarlet reporter for subsequent recombinant virus purification. For each insert, a P2A sequence was introduced between viral gene (S or N) and the reporter sequence for self-cleavage. (FIG. 1B) Schematic illustration of generation of recombinant MVA expressing SARS-CoV2 S and N (MVA-S+N). BHK-cells were infected with wild-type MVA (MOI: 0.01) for 2 hours, followed by co-transfecting cells with pLW17-S-mNeonGreen and pLW9-N-mScarlet plasmids. MVA-S+N were generated through homologous recombination and harvested 48 hours after transfection. (FIG. 1C) Confirmation for co-expression of the two reporters in the transfected cells by fluorescence microscope. Left: mNeonGreen (S), middle: mScarlet (N), right: overlay. (FIG. 1D) Microscopic confirmation of cells co-expressing mNeonGreen (S) and mScarlet (N) after FACS sorting. Transfected cells were subject to FACS sorting for mNeonGreen and mScarlet double positive population. Sorted cells were analyzed by fluorescence microscope. Left: mNeonGreen (S), middle: mScarlet (N), right: overlay. (FIG. 1E) Western blot (WB) confirmation of SARS-CoV2 S and N protein expression in cells infected with the vaccine. BHK-21 cells were infected with purified MVA-S+N for 48 hours. Proteins were extracted from the infected cells for WB analysis using specific antibody for S (GTX632604) or N (MA5-29981).

FIGS. 2A-2E. Vaccine-induced antibody response in mice following I.M. immunization. (FIG. 2A) Study design and vaccination timeline. 2 groups of WT BALB/c mice (n=5) were prime-boost immunized with mock (PBS) or MVA-S+N (10⁷ pfu) at week 0 and 3 via i.m. route. One week after prime vaccination (week 1), serum samples were collected for analysis of antibody response. Two weeks after boost vaccination (week 5), mice were euthanized and vaccine-induced humoral and cellular immune responses were analyzed. (FIG. 2B) S-specific (left) and N-specific (right) binding IgG in sera of the vaccinated mice (blue) compared to the control mice (red) after prime or boost vaccination. Antigen-specific binding IgG was measured by ELISA and the data are shown as OD450 values (initial serum dilution: 1:30). ELISA was conducted in duplicate and mean OD value for each sample was used. (FIG. 2C) Serum levels of S-specific and N-specific binding IgG in mice following boost (week 5) vaccination. Binding IgG in serially diluted sera is shown as OD450 nm values (mean±SD) for 5 mice of each group. (FIG. 2D) Serum SARS-CoV2 neutralizing activity was measured by Plaque Reduction Neutralization Test (PRNT). PRNT80 titers are shown. Negative and positive controls are included. (FIG. 2E) S and N binding IgG and IgA in BAL measured by ELISA (no dilution). *p<0.05; **p<0.01, ***p<0.001; unpaired student's t test; Mean (B, D. E), Mean±SD (C).

FIGS. 3A-3B. Vaccine-induced T-cell response in spleen following I.M. immunization. (FIG. 3A) ELLISPOT quantification of vaccine-specific T cells. Mouse splenocytes were ex vivo stimulated with overlapping peptide pools spanning SARS-CoV2 N protein or S protein for 20 hours. Spot forming cells (SFC) were measured by IFN-γ ELISPOT. Data are shown as # of SFC per 10⁶ splenocytes. (FIG. 3B) Measurement of vaccine-specific T cells by intracellular cytokine staining (ICS) and flow cytometry. Mouse splenocytes were ex vivo stimulated with overlapping peptide pools spanning SARS-CoV2 N protein or S protein for 5 hours. In the final 4 hours of stimulation, protein transport inhibitors (Golgi-stop/Golgi-plug) were added to the culture. Cells were then subject to ICS and flow cytometric analysis to measure IFN-γ+CD4 and CD8 T cells. *p<0.05; **p<0.01; unpaired student's t test; Mean±SD (A, B).

FIGS. 4A-4F. Vaccine-induced cellular immunity and antibody response in mouse lung after intranasal immunization. (FIG. 4A) Study design and vaccination timeline. 2 groups of WT BALB/c mice (n=5) were prime-boost immunized with mock (PBS) or MVA-S+N (10⁷ pfu) at week 0 and 3 via i.n. route. Two weeks after boost vaccination (week 5), mice were euthanized and vaccine-induced cellular immunity in lung tissues and antibody levels in BAL were analyzed. (FIG. 4B) Phenotypic analysis of immune cells in lung. Lung tissues of similar sizes were collected from mice and single-cell suspensions were prepared. Total cell numbers were counted and normalized to tissue weights. Cells were stained with live/dead viability dye, mouse CD45, CD3, CD4, CD8, and CD19, followed by flow cytometric analysis. Gating strategies for identifying CD45+ leukocytes, CD3+ T cells, CD3+CD8+ T cells, CD3+CD4+ T cells, and CD3−CD19+ B cells are shown. (FIG. 4C) Comparison of cell numbers for each cell type between the mock and vaccine group. (FIGS. 4D, 4E) Measurement of vaccine-specific CD4 and CD9 T cells expressing granzyme B (GZMB) by intracellular cytokine staining (ICS) and flow cytometric analysis. Lung single-cell suspensions were ex vivo stimulated with overlapping peptide pools spanning SARS-CoV2 N protein or S protein for 5 hours in the presence of protein transport inhibitors (Golgi-stop/Golgi-plug). Cells were then subject to ICS and flow cytometric analysis to measure GZMB-expressing CD8 and CD4 T cells. (D) Representative flow cytometric plots showing GZMB expression in CD8 T cells with short-term peptide re-stimulation. Negative control (mock stimulation) and positive control (PMA/ionomycin stimulation) are also shown. (E) Comparison of % GZMB-expressing CD8 T cells or CD4 T cells between the vaccinated and control. (F) Comparison of S- and N-specific binding IgG or IgA in BAL between mock and vaccine group. Binding IgG and IgA in BAL was measured by ELISA. *p<0.05; **p<0.01, ***p<0.001; unpaired student's t test; Mean (C, FIGS. 4E, 4F).

FIGS. 5A, 5B. In. immunization induces immune control of SARS-CoV-2 in the lung of mice following viral challenge. (FIG. 5A) Study design and timeline for vaccination and viral challenge. 2 groups of WT BALB/c mice (n=5) were prime-boost immunized with mock (PBS) or MVA-S+N (10⁷ pfu) at week 0 and 3 via i.n. route. Two weeks after boost vaccination (week 5), mice were intranasally challenged with mouse-adapted SARS-CoV2 strain (TCID: 2×10⁴). Two days after viral challenge, all mice were euthanized and vaccine-induced viral control in lung were analyzed. (FIG. 5B) Measurement of SARS-CoV2 viral RNAs in lung by quantitative PCR. Total RNA was extracted from the collected lung tissue. Viral RNAs (S, E, RdRp) and mouse GAPDH were quantified by q-PCR. Individual viral RNA was normalized to GAPDH and compared between the vaccine and mock group. Normalized PCR data are shown as fold change in RNA copies relative to those of the mock group. ***p<0.001; ****p<0.0001; unpaired student's t test; Mean (B).

FIG. 6. I.n. immunization diminishes SARS-CoV-2-induced inflammation in the mouse lung. Lung tissue RNAs collected from mock or MVA-vaccinated, SARS-CoV-2 challenged mice as described in FIGS. 5A,5B were subjected to qPCR quantification of host inflammatory genes. Lung tissue RNAs from unchallenged mice (n=5) were used as baseline control. Individual viral RNA was first normalized to GAPDH and then compared with the un-challenged control. The data are shown as fold change (mean) in RNA copies relative to those of the un-challenged control group. *p<0.05; **p<0.01; n.s.: non-significant; unpaired student's t test.

FIGS. 7A, 7B. Durability of vaccine-induced antibody response in mouse sera following I.M. immunization. Kinetics of S-specific (FIG. 7A) and N-specific (FIG. 7B) binding IgG in mouse sera of vaccinated (red) and control (grey) mice after boost vaccination. Antigen-specific binding IgG was measured by ELISA and the data are shown as OD450 values (Mean±SD) (serum dilution: 1:60). ELISA was conducted in duplicate.

FIG. 8. Frequencies of immune cells in mouse lung after i.m. immunization. Cells were collected from lung tissues and single-cell suspensions were prepared. Cells were stained for live/dead viability, mouse CD45, CD3, CD4, CD8, and CD19. Mouse leukocytes (CD45+), B cells (CD3−CD19+), CD3+CD4+ T cells, and CD3+CD8+ T cells were measured by flow cytometry. Shown are comparison of % leukocytes, CD4+ T cells, CD8+ T cells, and B cells in lung tissues of vaccinated (red) and control (black) mice (Mean). No statistical difference was observed between the two groups.

FIGS. 9A, 9B. SARS-CoV2 viral loads in lung in intramuscularly (i.m.) immunized, virally challenged mice. (FIG. 9A) Study design and timeline for i.m. vaccination and viral challenge. 2 groups of WT BALB/c mice (n=5) were prime-boost immunized with mock (PBS) or MVA-S+N (10⁷ pfu) at week 0 and 3 via i.m. Two weeks after boost vaccination (week 5), mice were intranasally challenged with mouse-adapted SARS-CoV2 strain (TCID: 2×10⁴). Two days after viral challenge, all mice were euthanized and vaccine-induced viral control in lung were analyzed. (FIG. 9B) Total RNA was extracted from the collected lung tissue. Viral RNAs (S, E, RdRp) and mouse GAPDH were quantified by q-PCR. Individual viral RNA was normalized to GAPDH and compared between the vaccine and mock group. Normalized PCR data are shown as fold change in RNA copies relative to those of the mock group (Mean).

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “antigen” refers to a molecule containing one or more epitopes (either linear, conformational or both) that will stimulate a host's immune-system to make a humoral and/or cellular antigen-specific response. The term is used interchangeably with the term “immunogen.” Normally, a B-cell epitope will include at least about 5 amino acids but can be as small as 3-4 amino acids. A T-cell epitope, such as a CTL epitope, will include at least about 7-9 amino acids, and a helper T-cell epitope at least about 12-20 amino acids. Normally, an epitope will include between about 7 and 15 amino acids, such as, 9, 10, 12 or 15 amino acids. The term includes polypeptides, which include modifications, such as deletions, additions and substitutions (generally conservative in nature) as compared to a native sequence, so long as the protein maintains the ability to elicit an immunological response, as defined herein. These modifications may be deliberate, as through site-directed mutagenesis, or may be accidental, such as through mutations of hosts, which produce the antigens.

As used herein, the term “immunological response” refers to an antigen or composition is the development in a subject of a humoral and/or a cellular immune response to an antigen present in the composition of interest. For purposes of the present disclosure, a “humoral immune response” refers to an immune response mediated by antibody molecules, while a “cellular immune response” is one mediated by T-lymphocytes and/or other white blood cells. One important aspect of cellular immunity involves an antigen-specific response by cytolytic T-cells (CTLs). CTLs have specificity for peptide antigens that are presented in association with proteins encoded by the major histocompatibility complex (MHC) and expressed on the surfaces of cells. CTLs help induce and promote the destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of cytokines, chemokines and other such molecules produced by activated T-cells and/or other white blood cells, including those derived from CD4+ and CD8+ T-cells. Hence, an immunological response may include one or more of the following effects: the production of antibodies by B-cells; and/or the activation of suppressor T-cells and/or gamma-delta T-cells directed specifically to an antigen or antigens present in the composition or vaccine of interest. These responses may serve to neutralize infectivity, and/or mediate antibody-complement, or antibody dependent cell cytotoxicity (ADCC) to provide protection to an immunized host. Such responses can be determined using standard immunoassays and neutralization assays, well known in the art.

As used herein, the term an “immunogenic composition” refers to a composition that comprises an antigenic molecule where administration of the composition to a subject results in the development in the subject of a humoral and/or a cellular immune response to the antigenic molecule of interest.

As used herein, the term “substantially purified” refers to isolation of a substance (compound, polynucleotide, protein, polypeptide, polypeptide composition) such that the substance comprises the majority percent of the sample in which it resides. Typically, in a sample a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

As used herein, the term “coding sequence” or a sequence which “encodes” a selected polypeptide, refers to a nucleic acid molecule that is transcribed (in the case of DNA) and translated (in the case of mRNA) into a polypeptide in vivo when placed under the control of appropriate regulatory sequences (or “control elements”). The boundaries of the coding sequence are determined by a start codon at the 5′ (amino) terminus and a translation stop codon at the 3′ (carboxy) terminus. A coding sequence can include, but is not limited to, cDNA from viral, prokaryotic or eukaryotic mRNA, genomic DNA sequences from viral or prokaryotic DNA, and even synthetic DNA sequences. A transcription termination sequence may be located 3′ to the coding sequence.

As used herein, the term “control elements”, includes, but is not limited to, transcription promoters, transcription enhancer elements, transcription termination signals, polyadenylation sequences (located 3′ to the translation stop codon), sequences for optimization of initiation of translation (located 5′ to the coding sequence), and translation termination sequences, and/or sequence elements controlling an open chromatin structure see e.g., McCaughan et al. (1995) PNAS USA 92:5431-5435; Kochetov et al (1998) FEBS Letts. 440:351-355.

As used herein, the term “nucleic acid” includes, but is not limited to, prokaryotic sequences, eukaryotic mRNA, cDNA from eukaryotic mRNA, genomic DNA sequences from eukaryotic (e.g., mammalian) DNA, and even synthetic DNA sequences. The term also captures sequences that include any of the known base analogs of DNA and RNA.

As used herein, the term “operably linked” refers to an arrangement of elements wherein the components so described are configured so as to perform their usual function. Thus, a given promoter operably linked to a coding sequence is capable of effecting the expression of the coding sequence when active. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

As used herein, the term “recombinant” refers to a polynucleotide of genomic, cDNA, semisynthetic, or synthetic origin which, by virtue of its origin or manipulation: (1) is not associated with all or a portion of the polynucleotide with which it is associated in nature; and/or (2) is linked to a polynucleotide other than that to which it is linked in nature. The term “recombinant” as used with respect to a protein or polypeptide means a polypeptide produced by expression of a recombinant polynucleotide. “Recombinant host cells,” “host cells,” “cells,” “cell lines,” “cell cultures,” and other such terms denoting prokaryotic microorganisms or eukaryotic cell lines cultured as unicellular entities, are used interchangeably, and refer to cells which can be, or have been, used as recipients for recombinant vectors or other transfer DNA, and include the progeny of the original cell which has been transfected. It is understood that the progeny of a single parental cell may not necessarily be completely identical in morphology or in genomic or total DNA complement to the original parent, due to accidental or deliberate mutation. Progeny of the parental cell which are sufficiently similar to the parent to be characterized by the relevant property, such as the presence of a nucleotide sequence encoding a desired peptide, are included in the progeny intended by this definition, and are covered by the above terms.

Techniques for determining amino acid sequence “similarity” are well known in the art. In general, “similarity” means the exact amino acid to amino acid comparison of two or more polypeptides at the appropriate place, where amino acids are identical or possess similar chemical and/or physical properties such as charge or hydrophobicity. A so-termed “percent similarity” then can be determined between the compared polypeptide sequences. Techniques for determining nucleic acid and amino acid sequence identity also are well known in the art and include determining the nucleotide sequence of the mRNA for that gene (usually via a cDNA intermediate) and determining the amino acid sequence encoded thereby and comparing this to a second amino acid sequence. In general, “identity” refers to an exact nucleotide to nucleotide or amino acid to amino acid correspondence of two polynucleotides or polypeptide sequences, respectively.

Two or more polynucleotide sequences can be compared by determining their “percent identity.” Two or more amino acid sequences likewise can be compared by determining their “percent identity.” The percent identity of two sequences, whether nucleic acid or peptide sequences, is generally described as the number of exact matches between two aligned sequences divided by the length of the shorter sequence and multiplied by 100. An approximate alignment for nucleic acid sequences is provided by the local homology algorithm of Smith and Waterman, Advances in Applied Mathematics 2:482-489 (1981). This algorithm can be extended to use with peptide sequences using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986), relevant portion incorporated herein by reference. Suitable programs for calculating the percent identity or similarity between sequences are generally known in the art.

As used herein, a polypeptide or peptide “variant” has at least 90%, 91%, 92%, 93%, 94%, 95%, 96%, 97%, 98%, 99%, or 100% or sequence identity with the amino acid sequence set forth in any one of SEQ ID NOS of the amino acid sequences disclosed herein. The polypeptide or peptide “variant” disclosed herein may have one or more amino acids deleted or substituted by different amino acids. It is well understood in the art that some amino acids may be substituted or deleted without changing biological activity of the peptide (conservative substitutions). Suitably, the variant has at least 90%, 91%, 92%, 93%, 94%, 95%, 96%, 97%, 98%, 99%, or 100% of the biological activity of the isolated polypeptide or peptide of any one of SEQ ID NOS of the amino acid sequences disclosed herein. In particular embodiments, the variant comprises, or is capable of forming antigenic proteins or polypeptides capable of triggering an immune response, whether humoral and/or cellular.

Terms used generally herein to describe sequence relationships between respective proteins and nucleic acids include “comparison window”, “sequence identity”, “percentage of sequence identity” and “substantial identity”. Because respective nucleic acids/proteins may each comprise (1) only one or more portions of a complete nucleic acid/protein sequence that are shared by the nucleic acids/proteins, and (2) one or more portions which are divergent between the nucleic acids/proteins, sequence comparisons are typically performed by comparing sequences over a “comparison window” to identify and compare local regions of sequence similarity. A “comparison window” refers to a conceptual segment of typically 6, 9 or 12 contiguous residues that is compared to a reference sequence. The comparison window may comprise additions or deletions (i.e., gaps) of about 20% or less as compared to the reference sequence for optimal alignment of the respective sequences. Optimal alignment of sequences for aligning a comparison window may be conducted by computerized implementations of algorithms (Geneworks program by Intelligenetics; GAP, BESTFIT, FASTA, and TFASTA in the Wisconsin Genetics Software Package Release 7.0, Genetics Computer Group, 575 Science Drive Madison, Wis., USA, incorporated herein by reference) or by inspection and the best alignment (i.e. resulting in the highest percentage homology over the comparison window) generated by any of the various methods selected. Reference also may be made to the BLAST family of programs as for example disclosed by Altschul et al., 1997, Nucl. Acids Res. 25 3389, which is incorporated herein by reference. A detailed discussion of sequence analysis can be found in Unit 19.3 of CURRENT PROTOCOLS IN MOLECULAR BIOLOGY Eds. Ausubel et al. (John Wiley & Sons Inc NY, 1995-2015), relevant portions incorporated herein by reference.

The term “sequence identity” is used herein in its broadest sense to include the number of exact nucleotide or amino acid matches having regard to an appropriate alignment using a standard algorithm, having regard to the extent that sequences are identical over a window of comparison. Thus, a “percentage of sequence identity” is calculated by comparing two optimally aligned sequences over the window of comparison, determining the number of positions at which the identical nucleic acid base (e.g., A, T, C, G, I) occurs in both sequences to yield the number of matched positions, dividing the number of matched positions by the total number of positions in the window of comparison (i.e., the window size), and multiplying the result by 100 to yield the percentage of sequence identity. For example, “sequence identity” may be understood to mean the “match percentage” calculated by the DNASIS or equivalent computer program (Version 2.5 for windows; available from Hitachi Software engineering Co., Ltd., South San Francisco, Calif., USA), relevant portions incorporated herein by reference.

The invention also provides fragments of the isolated peptide disclosed herein. In some embodiments, fragments may comprise, consist essentially of, or consist of 50%, 55%, 60%, 65%, 70%, 75%, 80%, 85%, 90%, 91%, 92%, 93%, 94%, 95%, 96%, 97%, 98% or 99% identity with any one of the amino acid sequences disclosed herein. In particular embodiments, the fragments comprise, or are capable of forming antigenic proteins or polypeptides capable of triggering an immune response, whether humoral and/or cellular.

Suitably, the fragments are antigenic proteins or polypeptides capable of triggering an immune response, whether humoral and/or cellular. Preferably, the fragment has at least 50%, 55%, 60%, 65%, 70%, 75%, 80%, 85%, 90%, 91%, 92%, 93%, 94%, 95%, 96%, 97%, 98% or 99% of the biological activity of the isolated peptide of any one of the amino acid sequences disclosed herein.

Derivatives of the isolated peptide disclosed herein are also provided. As used herein, “derivative” proteins or peptides have been altered, for example by conjugation or complexing with other chemical moieties, by post-translational modification (e.g. phosphorylation, ubiquitination, glycosylation), chemical modification (e.g. cross-linking, acetylation, biotinylation, oxidation or reduction and the like), conjugation with labels (e.g. fluorophores, enzymes, radioactive isotopes) and/or inclusion of additional amino acid sequences as would be understood in the art.

In this regard, the skilled person is referred to Chapter 15 of CURRENT PROTOCOLS IN PROTEIN SCIENCE, Eds. Coligan et al. (John Wiley & Sons NY 1995-2015), or equivalent, for more extensive methodology relating to chemical modification of proteins, relevant portions incorporated herein by reference. Additional amino acid sequences may include fusion partner amino acid sequences which create a fusion protein. By way of example, fusion partner amino acid sequences may assist in detection and/or purification of the isolated fusion protein. Non-limiting examples include metal-binding (e.g., polyhistidine) fusion partners, maltose binding protein (MBP), Protein A, glutathione S-transferase (GST), green fluorescent protein sequences (e.g., GFP), epitope tags such as myc, FLAG and haemagglutinin tags.

The isolated peptides, variant and/or derivatives of the present invention may be produced by any method known in the art, including but not limited to, chemical synthesis and recombinant DNA technology. Chemical synthesis is inclusive of solid phase and solution phase synthesis. Such methods are well known in the art, although reference is made to examples of chemical synthesis techniques as provided in Chapter 9 of SYNTHETIC VACCINES Ed. Nicholson (Blackwell Scientific Publications) and Chapter 15 of CURRENT PROTOCOLS IN PROTEIN SCIENCE Eds. Coligan et al., (John Wiley & Sons, Inc. NY USA 1995-2008). In this regard, reference is also made to International Publication WO 99/02550 and International Publication WO 97/45444. Recombinant proteins may be conveniently prepared by a person skilled in the art using standard protocols as for example described in Sambrook et al., MOLECULAR CLONING. A Laboratory Manual (Cold Spring Harbor Press, 1989), in particular Sections 16 and 17; CURRENT PROTOCOLS IN MOLECULAR BIOLOGY Eds. Ausubel et al., (John Wiley & Sons, Inc. NY USA 1995-2008), in particular Chapters 10 and 16; and CURRENT PROTOCOLS IN PROTEIN SCIENCE Eds. Coligan et al., (John Wiley & Sons, Inc. NY USA 1995-2008), in particular Chapters 1, 5 and 6, relevant portions incorporated herein by reference.

As used herein, the term a “vector” refers to a nucleic acid capable of transferring gene sequences to target cells (e.g., bacterial plasmid vectors, viral vectors, non-viral vectors, particulate carriers, and liposomes). Typically, “vector construct,” “expression vector,” and “gene transfer vector,” refers to any nucleic acid construct capable of directing the expression of one or more sequences of interest in a host cell. Thus, the term includes cloning and expression vehicles, as well as viral vectors. The term is used interchangeable with the terms “nucleic acid expression vector” and “expression cassette.”

As used herein, the term “subject” refers to any chordates, including, but not limited to, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered. The system described herein is intended for use in any of the above vertebrate species, since the immune systems of all of these vertebrates operate similarly.

As used herein, the terms “pharmaceutically acceptable” or “pharmacologically acceptable” refer to a material which is not biologically or otherwise undesirable, i.e., the material may be administered to an individual in a formulation or composition without causing any unacceptable biological effects or interacting in a deleterious manner with any of the components of the composition in which it is contained.

As used herein, the term “treatment” refers to any of (i) the prevention of infection or reinfection, as in a traditional vaccine, (ii) the reduction or elimination of symptoms, and (iii) the substantial or complete elimination of the pathogen in question. Treatment may be effected prophylactically (prior to infection) or therapeutically (following infection).

As used herein, the term “adjuvant” refers to a substance that non-specifically changes or enhances an antigen-specific immune response of an organism to the antigen. Generally, adjuvants are non-toxic, have high-purity, are degradable, and are stable. The recombinant adjuvant of the present invention meets all of these requirements; it is non-toxic, highly-pure, degradable, and stable. Adjuvants are often included as one component in a vaccine or therapeutic composition that increases the specific immune response to the antigen. However, the present invention includes a novel adjuvant that does not have to be concurrently administered with the antigen to enhance an immune response, e.g., a humoral immune response. Unlike the common principle of action of other immunologic adjuvants, such as: (1) increasing surface area of an antigen to improve the immunogenicity thereof; (2) causing slow-release of the antigen to extend the retention time of the antigen in tissue; or (3) promoting an inflammatory reaction to stimulate active immune response, the present invention targets the B cells directly to enhance the production of antibodies. Non-limiting examples of adjuvant for use with the present invention includes one or more adjuvants selected from alum, aluminum hydroxide, aluminum phosphate, calcium phosphate hydroxide, cytosine-guanosine oligonucleotide (CpG-ODN) sequence, granulocyte macrophage colony stimulating factor (GM-CSF), monophosphoryl lipid A (MPL), poly(I:C), MF59, Quil A, N-acetyl muramyl-L-alanyl-D-isoglutamine (MDP), FIA, montanide, poly (DL-lactide-coglycolide), squalene, glucopyranosyl lipid adjuvant (GLA), GLA-Alum, 3M-052, a glucopyranosyl lipid adjuvant GLA emulsion with squalene (GLA-SE), virosome, ASO3, ASO4, IL-1, IL-2, IL-3, IL-4, IL-5, IL-6, IL-7, IL-8, IL-10, IL-12, IL-15, IL-17, IL-18, STING, CD40L, pathogen-associated molecular patterns (PAMPs), damage-associated molecular pattern molecules (DAMPs), Freund's complete adjuvant, Freund's incomplete adjuvant, transforming growth factor (TGF)-beta antibody or antagonists, A2aR antagonists, lipopolysaccharides (LPS), Fas ligand, Trail, lymphotactin, Mannan (M-FP), APG-2, Hsp70 and Hsp90, pattern recognition receptor ligands, TLR3 ligands, TLR4 ligands, TLR5 ligands, TLR7/8 ligands, or TLR9 ligands.

As used herein, the term “effective dose” refers to that amount of an immunogenic peptide or fusion protein that includes the coronavirus antigens described herein. Further, the immunogenic peptide can be fused with another protein to express and/or display the antigenic epitope or to provide a fusion protein that is processed by antigen presenting cells for display in the context of MHC Class I and/or Class II protein. As described herein, the antigenic peptide can be fused to an N-terminal, C-terminal, and/or a loop formed between amino acid 74 and 82 to form a fusion protein that includes, e.g., a coronavirus Receptor Binding Motif (RBM) of the spike protein (S protein), a nucleocapsid protein (N protein), or both such as a SARS-CoV-2 spike protein, of the present invention sufficient to induce immunity, to prevent and/or ameliorate an infection or to reduce at least one symptom of an infection and/or to enhance the efficacy of another dose of a coronavirus. An effective dose may refer to the amount of the fusion protein sufficient to delay or minimize the onset of an infection. An effective dose may also refer to the fusion protein in an amount that provides a therapeutic benefit in the treatment or management of an infection. Further, an effective dose is the amount with respect to the fusion protein of the invention alone, or in combination with other therapies, that provides a therapeutic benefit in the treatment or management of an infection. An effective dose may also be the amount sufficient to enhance a subject's (e.g., a human's) own immune response against a subsequent exposure to an infectious agent. Levels of immunity can be monitored, e.g., by measuring amounts of neutralizing secretory and/or serum antibodies, e.g., by plaque neutralization, complement fixation, enzyme-linked immunosorbent, or microneutralization assay. In the case of a vaccine, an “effective dose” is one that prevents disease and/or reduces the severity of symptoms.

As used herein, the term “immune stimulator” refers to a compound that enhances an immune response via the body's own chemical messengers (cytokines). These molecules comprise various cytokines, lymphokines and chemokines with immunostimulatory, immunopotentiating, and pro-inflammatory activities, such as interferons, interleukins (e.g., IL-1, IL-2, IL-3, IL-4, IL-12, IL-13); growth factors (e.g., granulocyte-macrophage (GM)-colony stimulating factor (CSF)); and other immunostimulatory molecules, such as macrophage inflammatory factor, Flt3 ligand, B7.1; B7.2, etc. The immune stimulator molecules can be administered in the same formulation as the HBcAg-RBM fusion protein of the present invention, or can be administered separately. Either the protein or an expression vector encoding the protein can be administered to produce an immunostimulatory effect.

As used herein, the term “innate immune response stimulator” refers to agents that trigger the innate or non-specific immune response. The innate immune response is a nonspecific defense mechanism is able to act immediately (or within hours) of an antigen's appearance in the body and the response to which is non-specific, that is, it responds to an entire class of agents (such as oligosaccharides, lipopolysaccharides, nucleic acids such as the CpG motif, etc.) and does not generate an adaptive response, that is, they do not cause immune memory to the antigen. Pathogen-associated immune stimulants act through the Complement cascade, Toll-like Receptors, and other membrane bound receptors to trigger phagocytes to directly kill the perceived pathogen via phagocytosis and/or the expression of immune cell stimulating cytokines and chemokines to stimulate both the innate and adaptive immune responses. The present inventors take advantage of the innate immune response to help enhance the adaptive immune response by glycosylating to the antigens taught herein, thus enhancing antigen presentation and generation of both T and B cell-drive immune responses.

As used herein, the term “protective immune response” or “protective response” refers to an immune response mediated by antibodies or effector cells against an infectious agent, which is exhibited by a vertebrate (e.g., a human), which prevents or ameliorates an infection or reduces at least one symptom thereof.

As used herein, the term “antigenic formulation” or “antigenic composition” refers to a preparation which, when administered to a vertebrate, e.g., a mammal, induces an immune response.

As used herein, the terms “immunization” or “vaccine” are used interchangeably to refer to a formulation which contains the antigenic fusion protein(s) of the present invention, which is in a form that is capable of being administered to a vertebrate and which induces a protective immune response sufficient to induce immunity to prevent and/or ameliorate an infection and/or to reduce at least one symptom of an infection and/or to enhance the efficacy of another dose or exposure to the coronavirus. Typically, the vaccine comprises a conventional saline or buffered aqueous solution medium in which the composition of the present invention is suspended or dissolved. In this form, the composition of the present invention can be used conveniently to prevent, ameliorate, or otherwise treat an infection. Upon introduction into a host, the vaccine is able to provoke an immune response including, but not limited to, the production of antibodies and/or cytokines and/or the activation of cytotoxic T cells, antigen presenting cells, helper T cells, dendritic cells and/or other cellular responses. More particularly, in one specific embodiment, the present invention induces a cellular immune response without a significant, or any, humoral immune response.

The practice of the present invention employs, unless otherwise indicated, conventional methods of chemistry, biochemistry, molecular biology, immunology and pharmacology, within the skill of the art. Such techniques are explained fully in the literature. See, e.g., Remington's Pharmaceutical Sciences, 18th Edition (Easton, Pa.: Mack Publishing Company, 1990); Methods In Enzymology (S. Colowick and N. Kaplan, eds., Academic Press, Inc.); and Handbook of Experimental Immunology, Vols. I-IV (D. M. Weir and C. C. Blackwell, eds., 1986, Blackwell Scientific Publications); Sambrook, et al., Molecular Cloning: A Laboratory Manual (2nd Edition, 1989); Short Protocols in Molecular Biology, 4th ed. (Ausubel et al. eds., 1999, John Wiley & Sons); Molecular Biology Techniques: An Intensive Laboratory Course, (Ream et al., eds., 1998, Academic Press); PCR (Introduction to Biotechniques Series), 2nd ed. (Newton & Graham eds., 1997, Springer Verlag); Fundamental Virology, Second Edition (Fields & Knipe eds., 1991, Raven Press, New York), relevant portion incorporated herein by reference.

Current SARS-CoV-2 vaccine approaches principally focus on targeting the viral spike protein (S), or its receptor-binding domain (RBD) in particular, and the major goal is eliciting protective neutralizing antibodies [23-25]. Other than neutralizing antibodies, virus-induced T cells have also been indicated to play a role in control of coronavirus infections [10, 26-30]. Previous studies on SARS-CoV-1 suggested that, compared to virus-induced antibodies which were relatively short-lived [31], virus-specific T cells could be maintained for longer periods of time [32, 33]. Thus, vaccine strategies aiming for inducing robust T-cell responses, especially in the respiratory tract, apart from humoral immunity will likely generate important information for pan-coronavirus vaccine development. Among the SARS-CoV-2 viral proteins, the S protein contains T-cell epitopes and stimulates abundant S-specific T-cell responses in the infected individuals [34-36]. In addition to S, the viral nucleoprotein (N) also contains critical T-cell epitopes but demonstrates fewer mutations and is more conserved across different coronaviruses [37]. Evidence suggests that functional N-specific T-cell responses could be long-lasting [38] and correlates with viral control [30]. Thus, N protein may represent another promising immunogen for incorporation in SARS-CoV-2 vaccine design [39]. Currently, another key question related to host immunity to SARS-CoV-2 and vaccine development is whether or not vaccine-elicited cellular immunity could confer immune control of SARS-CoV-2 in the absence of neutralizing antibodies.

In this study, the inventors demonstrate a multigenic SARS-CoV2 vaccine approach based on the modified vaccinia ankara (MVA) vector that expresses both viral N and S proteins (MVA-S+N). It is demonstrated herein that the vaccine was immunogenic in mice and intramuscular immunization with the vaccine induced robust systemic T-cell responses and binding antibodies specific to both antigens, whereas no neutralizing activity against SARS-CoV-2 was induced by the vaccine. Of particular interest, intranasal immunization with the vaccine elicited strong local N- and S-specific T-cell responses in the lung, including granzyme B-expressing cytotoxic CD8+ T cells. In a SARS-CoV-2 challenge model, where BALB/c mice were intranasally infected with a mouse-adapted SARS-CoV-2 strain, it was observed that, compared to the mock immunized animals, intranasal immunization with the vaccine conferred significant protection against SARS-CoV-2 infection in the absence of neutralizing activity as evidenced by diminished viral loads and virus-induced inflammation in the lung, whereas there was no evidence of protective effect following intramuscular immunization with the same vaccine.

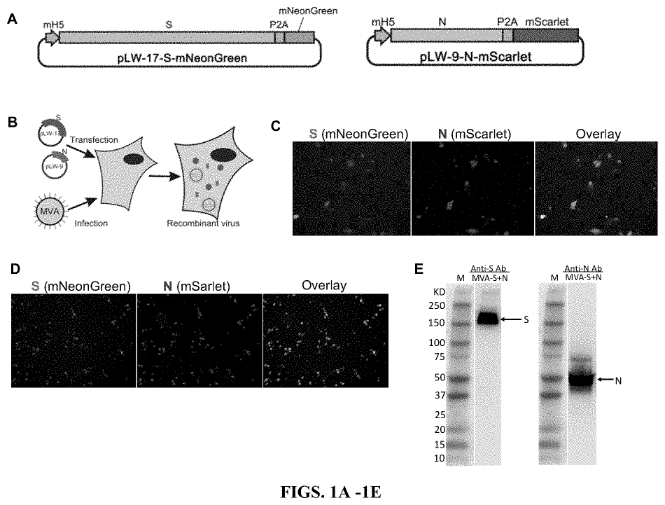

Design, generation and in vitro characterization of recombinant MVA-S+N. MVA is a large poxviral vector and can accommodate multiple transgenes for expression [40]. A variety of transfer plasmids have been developed for the generation of recombinant MVA through homologous recombination, including pLW17 and pLW9 [41]. To generate recombinant MVA expressing SARS-CoV-2 N and S (MVA-S+N), wild-type (WT) full-length S and N genes (2019-nCoV/USA-WA1/2020) [42] were respectively cloned into the pLW17 and pLW9 transfer plasmids to construct pLW17-S and pLW9-N (FIG. 1A). To aid subsequent recombinant virus purification, the S and N sequences were linked to mNeonGreen and mScarlet reporter, respectively, via a self-cleavage site P2A [43]. BHK-21 cells were first infected with WT MVA for 2 hours, followed by transfection with the two transfer constructs (pLW17-S-mNeonGreen and pLW-9-mScarlet). 48 hours after transfection, MVA-S+N in cells were generated through homologous recombination (FIG. 1B). As shown in FIG. 1C, cells co-expressing mNeonGreen and mScarlet reporters were identified by fluorescence microscope. To purify the recombinant virus, two rounds of live cell sorting using flow cytometry were performed to isolate double-positive cells. Microscopic analysis confirmed double positivity of cells after sorting (FIG. 1D). Recombinant MVA-S+N from lysates of the sorted cells was subject to several rounds of plaque purification [40]. The vaccine candidate was then propagated, concentrated and titrated as previously reported [40]. Before animal immunization, efficient expression of both SARS-CoV-2 S and N proteins in BHK-21 cells following MVA-S+N infection was confirmed by western blot analysis (FIG. 1E).

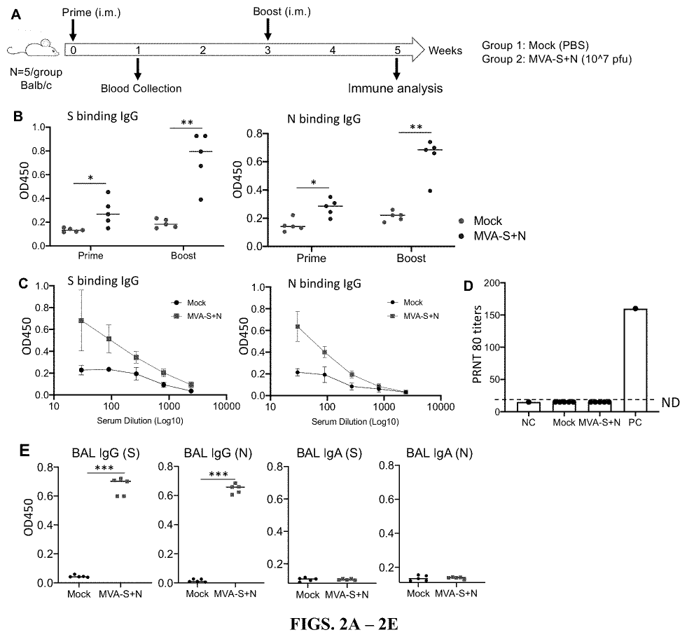

Intramuscular immunization with MVA-S+N induces systemic and local SARS-CoV-2 specific binding antibodies without neutralizing activities. Immunogenicity of the MVA-S+N vaccine was evaluated in WT BALB/c mice. Two groups of mice were vaccinated with PBS (mock) or MVA-S+N. Vaccination was given intramuscularly (i.m.) at week 0 (prime) and week 3 (booster) (FIG. 2A). The vaccine dose was 10⁷ pfu/mouse according to previous studies [44, 45]. One week after prime vaccination, blood was collected for analysis of antibody responses; two weeks after boost vaccination, mice were euthanized and multiple specimens (blood, spleen and lung/bronchoalveolar lavage) were collected for analysis of vaccine-induced, systemic and local humoral and cellular immune responses (FIG. 2A).

First, vaccine-induced binding IgG in sera was measured by ELISA. The data showed that compared to mock, the vaccine was able to rapidly induce detectable, albeit at low levels, binding IgG seven days after the 1^{st} vaccination; both anti-S and anti-N binding IgG were readily detectable at comparable levels (FIG. 2B). Compared to prime vaccination, boost vaccination significantly enhanced the levels of both anti-S and anti-N binding IgG in the sera (FIG. 2B). For sera collected after booster immunization, they were also serially diluted (1:3) and the binding IgG in them was similarly measured by ELISA (FIG. 2C). The data confirmed that compared to mock, the vaccine induced binding IgG responses to both S and N antigens (FIG. 2C). In addition, these studies monitoring the kinetics of antibody responses indicated that vaccine-induced S- and N-specific binding IgG was fairly durable and remained readily detectable 11 weeks post boost vaccination (FIGS. 7A, 7B). These data suggest that MVA-S+N vaccine is immunogenic in mice and able to induce systemic N- and S-specific binding IgG at comparable levels in sera.

Vaccine-induced neutralizing activity was determined by the Plaque Reduction Neutralizing Test (PRNT) [46] using the live SARS-CoV2 virus (2019-nCoV/USA-WA1/2020). As expected, while the vaccine induced significant binding IgG to the S protein in sera, no neutralizing activity was detected in any of the vaccinated mice (FIG. 2D). The lack of vaccine-induced neutralizing antibodies was confirmed by a different SARS-CoV-2 live virus neutralization assay [47] and was consistent with previous findings that the pre-fusion stabilized mutations in the S gene is critical for the induction of neutralizing antibody response [18, 48-50].

The respiratory system including lung is the primary site of SARS-CoV2 infection and pathogenesis [51]. Induction of strong immune response in the lung is likely critical for the success of a SARS-CoV-2 vaccine. In the mouse immunogenicity study, following i.m. administration of the vaccine, bronchoalveolar lavage (BAL) was also collected from mice immediately following euthanasia to measure vaccine-induced antibody production in the lung. S- and N-specific binding IgG as well as IgA in the BAL were measured by ELISA. It was observed that i.m. administration of the vaccine induced significant levels of N- and S-specific binding IgG in the lung after boost vaccination (FIG. 2E). Unlike IgG, no IgA was detected in the BAL following i.m. vaccination (FIG. 2E). Together, these serological data indicate that i.m. immunization with the MVA-S+N vaccine induces both systemic and local anti-S and anti-N IgG responses in mice but no neutralizing activity was elicited.

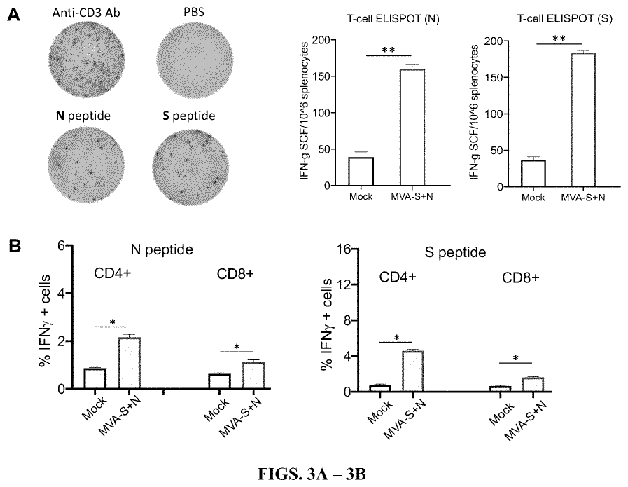

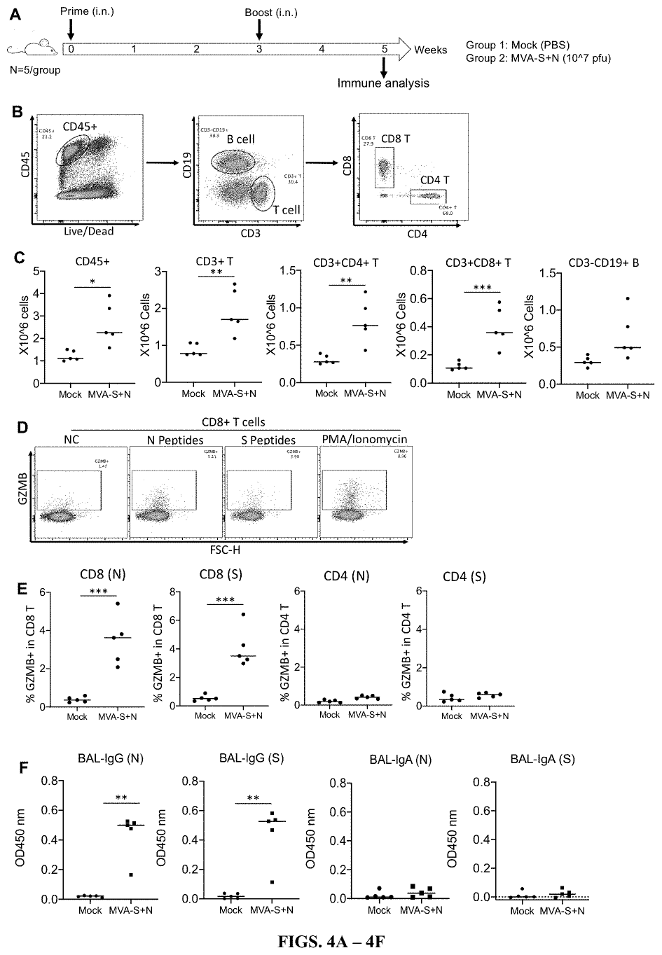

Intramuscular immunization induces robust N- and S-specific T cell responses in mouse spleen but not lung. Next, the inventors examined vaccine-induced, systemic T-cell responses in the spleen by IFN-γ ELISPOT. Splenocytes were re-stimulated with overlapping peptide pools spanning SARS-CoV2 N protein or S protein for 20 hours, and IFN-γ-producing T cells in splenocytes were quantified by ELISPOT based on spot forming counts (SFC). The data showed that, similar to antibody responses as described above, the MVA-S+N vaccine induced robust N- and S-specific T-cell responses in spleen, whereas very low basal levels of SFC were observed in the mock-vaccinated group (FIG. 3A).

To differentiate antigen-specific CD4 vs. CD8 T-cell response induced by the vaccine, splenocytes were also stimulated with overlapping peptide pools spanning SARS-CoV2 N protein or S protein for 5 hours in the presence of cytokine transport inhibitors and then subjected to intracellular IFN-γ cytokine staining (ICS) and flow cytometric analysis. In line with the T-cell ELISPOT results, ICS analysis showed that, compared to the mock-vaccinated group, MVA-S+N vaccine induced robust IFN-γ-expressing CD4+ and CD8+ T cells specific to both N and S proteins (FIG. 3B). Following i.m. immunization, the vaccine appeared to induce higher magnitudes of antigen-specific CD4 T-cell responses than CD8 T-cell responses for both S and N proteins in the spleen (FIG. 3B). In addition to the spleen, the inventors also measured local cellular response in the lung by flow cytometry (FIG. 8). While intramuscular immunization induced robust systemic T-cell responses in the spleen, it did not elicit increases in the frequencies of major immune cells, including CD45+ leukocytes, T cells, and B cells in the lungs (FIG. 8). In contrast to the cellular response, the vaccine induced significant and comparable levels of N- and S-specific binding IgG in the BAL (FIG. 2E), which is likely circulating from the blood. Together, these data indicate that i.m. immunization with MVA-S+N elicits robust S- and N-specific CD4 and CD8 T-cell response in the spleen, but not in the lung.

Intranasal immunization induces robust local cellular immune response in the lung. Although this initial finding indicated that the vaccine was immunogenic and i.m. immunization induced systemic binding IgG and T-cell responses, this immunization approach did not appear to induce strong cellular immunity in the mouse lung. Given that the respiratory system is the primary site of SARS-CoV-2 entry and replication, the inventors explored whether or not mucosal delivery of the vaccine could induce more robust cellular responses in the lung. Two groups of WT BALB/c mice were intranasally immunized with either PBS (mock control) or the MVA-S+N vaccine at week 0 (prime) and week 3 (boost). The vaccine dose (10⁷ pfu/mouse) was the same as that used in i.m. immunization. Two weeks after boost immunization (week 5), all mice were euthanized, followed by sample collection and analysis of cellular and antibody responses in the lung (FIG. 4A).

The inventors first assessed pulmonary cellular immune responses by measuring the frequencies of leukocytes, including B cells, CD4 T cells and CD8 T cells, after vaccination. Single-cell suspensions were prepared from the lung tissues for phenotypic analysis. In FIG. 4B, the gating strategy was shown for the identification of leukocytes (CD45+), B cells (CD3−CD19+), CD4+ T cells (CD3+CD34+), and CD8+ T cells (CD3+CD8+) by flow cytometry. Total cell numbers for each population were quantified and normalized to the collected lung tissues. Compared to the mock group, MVA-S+N vaccination induced significantly increased numbers of total CD45+ leukocytes, CD4+ T cells, and CD8+ T cells in the lung. Among these cell populations, CD8+ T cells appeared to be most profoundly enhanced by vaccination (>3-fold increase compared to the mock group) (FIG. 4C). Vaccination also induced a trend of modest increase in B-cell numbers in the lung with no statistical significance detected (FIG. 4C).

Other than neutralizing antibodies, virus-specific T cells residing in the lung may play a critical role in the immune control of coronaviruses, including SARS-CoV-2 [26, 27]. The inventors therefore performed ex vivo peptide stimulation and intracellular cytokine staining (ICS) analysis to examine the magnitudes and functional characteristics of vaccine-specific T cells in the lung after vaccination. Next, it was determined if mucosal immunization with the vaccine induces cytotoxic potential of viral antigen-specific CD8 T cells in the lung. Single-cell suspensions from the lung tissues were ex vivo-stimulated with the overlapping peptide pools spanning SARS-CoV2 N protein or S protein for 5 hours, followed by cell surface staining for lineage markers and intracellular staining for mouse granzyme B (GZMB). Representative FACS plots using cells of the vaccinated mouse (FIG. 4D) showed that while there was basal level of GZMB in the un-stimulated CD8+ T cells (NC) (˜1.47%), stimulation of cells with N or S peptides induced significant increase in GZMB+ CD8 T cells (N peptides: 5.11%; S peptides: 3.98%) (FIG. 4D). Cumulative analysis demonstrated that compared to the mock group, intranasal MVA-S+N vaccination induced robust GZMB-expressing CD8+ T cells specific for both viral antigens (N and S) in the lung, indicating cytotoxic potential of these CD8+ T cells (FIG. 4E). In the lungs, compared to CD8+ T cells, the total number of CD4+ T cells was higher (FIG. 4E). Despite a small fraction of CD4+ T cells expressed GZMB expression upon peptide re-stimulation, the frequency of GZMB-expressing, SARS-CoV-2-specific CD4 T cells in the lung was much lower and not enhanced by vaccination in these mice (FIG. 4E).

Similar to i.m. vaccination (FIG. 2E), vaccine-induced antibody levels in the BAL following i.n vaccination were also measured. The data showed that i.n. vaccination induced significant levels of N- and S-specific binding IgG in the BAL and no IgA was induced (FIG. 4F), consistent with the results of i.m. vaccination where slightly higher levels of N- and S-specific binding IgG were also detected in the BAL (FIG. 2E). Together, these data indicate that the MVA-S+N vaccine is also immunogenic via mucosal (intranasal) immunization and induces robust cellular immunity in the lung. While both i.n. and i.m. immunizations induced comparable levels of binding IgG in the BAL, the T-cell immunity induced by the two approaches is distinct with i.n. immunization being able to elicit robust T-cell response in the lung.

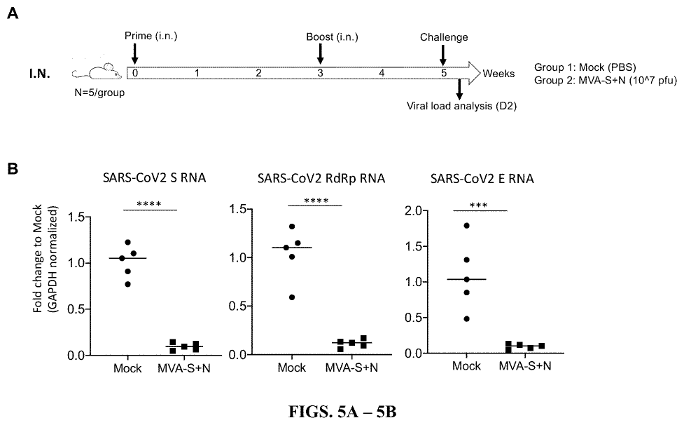

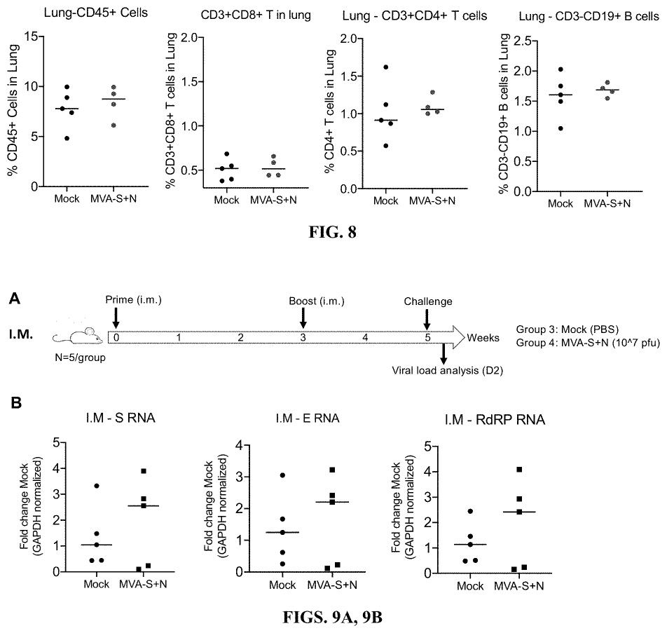

Intranasal immunization induces immune protection and reduces viral loads in the lung after SARS-CoV-2 challenge. To evaluate the protective efficacy of the vaccine following i.n. and i.m. immunization, a challenge study was performed with BALB/c mice using a mouse-adapted SARS-CoV2 strain. To avoid inter-experimental variation, immunization and viral challenge were conducted in parallel for i.n.- and i.m.-vaccinated mice. Study design and immunization schedule were the same as those described in the immunogenicity studies. As outlined in FIG. 5A and FIG. 9A, two groups of BALB/c mice (n=5) received PBS (mock) or MVA-S+N vaccine via i.n. immunization at week 0 and 3, respectively. Another two groups of mice (n=5) received the same vaccinations but via i.m. administration. Vaccination timelines and doses were identical between the i.n. and i.m. groups. On week 5 (two weeks after boost vaccination), all mice were intranasally challenged with a mouse-adapted SARS-CoV2 strain (TCID50: 2×10⁴ pfu per mouse). Mice were euthanized two days after viral challenge for analysis of viral loads in the lung. Three different SARS-CoV2 viral RNAs (S, E, and RdRp) were examined in the lung tissues by qPCR to determine the viral loads. For i.n. immunization, it was observed that, compared to the mock group, vaccination significantly reduced the viral loads in all the vaccinated mice, based on all three viral RNAs examined (S: >11-fold; E: >12-fold; RdRp: >9-fold) (FIG. 5B). The difference between the vaccine and mock groups was statistically highly significant (p<0.0001 for S and E, p<0.001 for RdRp) (FIG. 5B). In contrast, no statistical difference in the viral loads was observed between the vaccine and mock groups following i.m. immunization (p=0.55 for S, p=0.74 for E, and p=0.4 for RdRp) (FIG. 9B). The distinct outcomes of vaccine-induced immune control of SARS-CoV-2 between the i.n. and i.m. immunizations correlated with the difference in cellular immunity in the lung elicited by these two approaches.

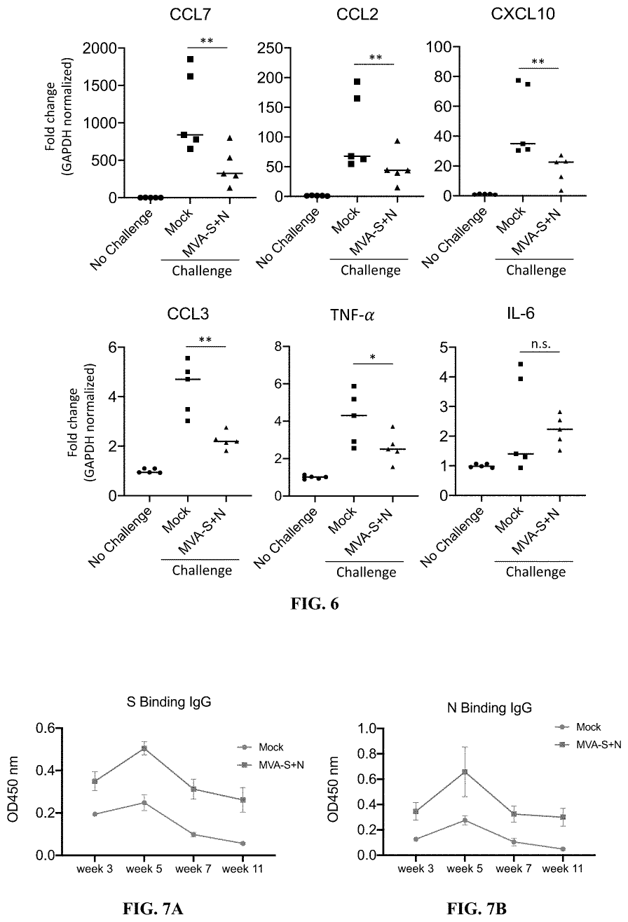

Intranasal immunization diminishes virus-induced inflammation in lung after SARS-CoV-2 challenge. In addition to the viral loads, infection-induced inflammation and pathology in the lung of mice following i.n. immunization and viral challenge were also examined. Lung tissue RNA samples collected from the two i.n. vaccination groups (mock and MVA-S+N) described above in FIGS. 5A, 5B, as well as from the unchallenged mice (as baseline control), were used. RNA samples were subjected to PCR quantification of host inflammatory genes related to lung pathology, including CCL2, CCL3, CCL7, CXCL10, TNF-α, and IL-6 (FIG. 6). The data showed that, compared to the unchallenged control, intranasal challenge with SARS-CoV-2 induced marked up-regulation of inflammatory gene expression. Among the genes examined, CCL7, CCL2 and CXCL10 were abundantly up-regulated (FIG. 6; top), and CCL3, TNF-α, and IL-6 were modestly or slightly up-regulated (FIG. 6; bottom). Of importance, compared to the mock-vaccinated, virally challenged group, i.n. immunization with the MVA-S+N vaccine significantly diminished the expression of 5 out of 6 of these genes (CCL2, CCL3, CCL7, CXCL10, TNF-α), except IL-6 (FIG. 6). The data are consistently the results of viral loads and support that the MVA-S+N vaccine can alleviate SARS-CoV-2-induced inflammation and pathology in the lung.

Recombinant vaccine construction. The spike (S) and nucleocapsid (N) genes of SARS-CoV2 were amplified from the infectious cDNA clone of 2019-nCoV/USA-WA1/2020 strain [62], fused with a gene cassette of porcine teschovirus-1 2A (P2A) and a fluorescent marker (S gene with mNeonGreen and N gene with mScarlet) (FIG. 1D). Gene insertions were respectively cloned to transfer plasmid pLW17 or pLW9 (kindly provided by Dr. Bernard Moss) by using NEBuilder HiFi DNA Assembly mix (Cat #: E2621; NEB) to generate plasmid constructs pLW17-S-mNeonGreen and pLW9-N-mScarlet.

Vaccine generation and purification. Recombinant MVA encoding SARS-CoV2 S and N genes were generated using a protocol as previously reported [63] with modifications. Briefly, monolayers of BHK-21 were grown in complete DMEM medium in six-well culture plates to 80% confluency. Cells were then infected with wild-type MVA (VR-1508; ATCC) at 0.01 multiplicity of infection (MOI) for 2 hours, followed by co-transfection with plasmids pLW17-S-mNeonGreen and pLW9-N-mScarlet using Lipofectamine 3000 Transfection Kit (Cat #: L3000-015; Invitrogen). 48 hours after transfection, cells co-expressing mNeonGreen and mScarlet in the culture plate were confirmed by fluorescence microscope (FIG. 1C). Cells were then harvested and sort purified for mNeonGreen and mScarlet double-positive population by the BD FACS Sorter (UTMB flow cytometry and cell-sorting core). Lysates of sorted cells were used to further purify recombinant MVA encoding both S and N (MVA-S+N) by using the plaque purification protocol as reported previously [63] (4-5 rounds based on mNeonGreen and mScarlet marker). Purified MVA-S+N virus was propagated in BHK-21 cells, concentrated, and titrated as previously reported [63].

Vaccine in vitro characterization. Purified MVA-S+N vaccine was first characterized in infected BHK-21 cells by using fluorescence microscope. Monolayers of BHK-21 cells at 80% confluency were infected with plaque purified MVA-S+N (MOI=1) for 48 hours. Co-expression of mNeonGreen and mScarlet in the infected cells was examined by fluorescence microscope. In addition, the vaccine was characterized for SARS-CoV2 S and N protein expression in the infected BHK-21 cells by western blot. Briefly, BHK-21 cells were infected with recombinant MVA-S+N (MOI=1) for 48 hours. Infected cells were lysed in RIPA buffer (Thermo Fisher Scientific) and kept on ice for 15 minutes. Cell lysates were centrifuged and the supernatants were collected for quantification of total protein concentration using Microplate BCA Protein Assay Kit (Pierce™, Thermo Fisher Scientific). Equivalent amounts of protein were separated by SDS-PAGE using precast 4-15% SDS polyacrylamide gels (Bio-Rad). Proteins were subsequently transferred onto a nitrocellulose membrane (Bio-Rad). The membrane was blocked in tris buffered saline (TBS) containing 0.05% Tween-20 (Thermo Fisher Scientific) and 5% (w/v) non-fat dried milk (Bio-Rad) for 1.5 hours at room temperature, followed by incubation with anti-SARS-CoV2 spike mouse mAb (GTX632604, GeneTex; 1:500) or anti-SARS-CoV2 nucleocapsid mouse mAb (MA5-29981, Invitrogen; 1:1000) for overnight at 4° C. After washing in TBST (3 times for 5 minutes), the membrane was incubated for 1 hour with HRP-linked anti-mouse IgG (7076S, Cell Signaling; 1:5000). The membrane was washed, and proteins were visualized using the ECL Western Blotting Substrate (Thermo Fisher Scientific).

Mouse immunization, sample collection and immunogenicity analysis. Animal study was conducted in accordance with the recommendations in the Guide for the Care and Use of Laboratory Animals of the National Institutes of Health. Animal protocol was approved by the Institutional Animal Care and Use Committee (IACUC) at the University of Texas Medical Branch. Animal study design and experimental timelines were summarized in different figures of the manuscript. Briefly, 6-week old female BALB/c mice were obtained from the Jackson Laboratories (Wilmington, Mass., USA) and were housed in the animal facility at the Medical Research Building of the University of Texas Medical Branch. Mice (5 per group) were immunized intramuscularly (i.m.) or intranasally (i.n.) with either PBS (50 μl) as the mock control or 10⁷ PFU MVA-S+N vaccine (50 μl) using a prime-boost approach at week 0 (prime) and week 3 (boost), respectively. For immunogenicity studies, blood/serum samples were collected from all mice one week after prime (1^{st}) vaccination to measure antibody response. Two weeks after the 2^{nd} vaccination (week 5), mice were euthanized. Blood/serum, spleen, and lung tissues were collected for immune analyses. Bronchoalveolar lavage (BAL) was also collected by washing the lung with 1 ml ice-cold Dulbecco's phosphate-buffered saline (DPBS) by using a blunt-ended needle as previously reported [64]. BAL was used for quantifying vaccine-induced antibody response in lung.

Binding IgG and IgA by ELISA. ELISA was used to measure N- and S-specific binding IgG and IgA in sera and in BAL. ELISA plates (Greiner bio-one) were coated with 1 μg/ml recombinant S (S1+S2-ECD; 40589-V08B1; Sino Biological) or N protein (40588-V08B; Sino Biological) in DPBS overnight at 4° C. Plates were washed three times with wash buffer (DPBS with 0.05% Tween 20), 5 minutes for each time, and then blocked with 8% FBS in DPBS for 1.5 hour at 37° C. Plates were washed and incubated with serially diluted sera in blocking buffer at 50 μl per well for 1 hour at 37° C. For quantification of binding antibodies in BAL, collected BAL fluids were used for incubation without dilution. ELISA was conducted in duplicate. Plates were again washed and incubated with horse radish peroxidase (HRP) conjugated anti-mouse IgG secondary antibody (Biolegend) (1:5000) for 1 hour at 37° C. After final wash, plates were developed using TMB 1-Component Peroxidase Substrate (Thermo Fisher), followed by termination of reaction using the TMB stop solution (Thermo Fisher). Plates were read at 450 nm wavelength within 30 min by using a Microplate Reader (BioTek).

Neutralizing assay. Neutralizing activity was examined by a standard Plaque Reduction Neutralization Test (PRNT) as previously reported [47, 53] with slight modifications. The assays were performed with Vero cells using live SARS-CoV-2 at BSL-3. In brief, sera were heat-inactivated and two-fold serially diluted (dilution range of 1:10 to 1:640), followed by inculcation with 100 PFU SARS-CoV2 (USA-WA1/2020) for 1 hour at 37° C. The serum-virus mixtures were placed onto Vero E6 cell monolayer in 6-well plates for incubation for 1 hour at 37° C., followed by addition of 2-ml overlay consisting of MEM with 1.6% agarose, 2% FBS and 1% penicillin-streptomycin to the cell monolayer. Cells were then incubated for 48 hours at 37° C., followed by staining with 0.03% liquid neutral red for 3-4 hours. Plaque numbers were counted and PRNT80 were calculated. Each serum was tested in duplicates.

IFN-γ ELISPOT. Millipore ELISPOT plates (Millipore Ltd, Darmstadt, Germany) were coated with anti-IFN-γ capture Ab (CTL, Cleveland, Ohio, USA) at 4° C. overnight. Splenocytes (0.25×10⁶) were stimulated in duplicates with SARS-CoV-2 S or N peptide pools (2 μg/ml, Miltenyi Biotec, USA) for 24 hours at 37° C. Splenocytes stimulated with anti-CD3 (1 μg/ml, e-Biosciences) or medium alone were used as controls. This was followed by incubation with biotin-conjugated anti-IFN-γ (CTL, Cleveland, Ohio, USA) for 2 hours at room temperature, and then alkaline phosphatase-conjugated streptavidin for 30 minutes. The plates were washed and scanned using an ImmunoSpot 4.0 analyzer and the spots were counted with ImmunoSpot software (Cellular Technology Ltd, Cleveland, Ohio) to determine the spot-forming cells (SFC) per 10⁶ splenocytes.

Intracellular Cytokine Staining (ICS) and Flow Cytometry. ICS was performed on splenocytes or lung tissue-isolated single-cell preparations. Cells (2×10⁶) were stimulated for 5 hours at 37° C. with 1 μg/ml SARS-CoV-2 S or N peptide pool (Miltenyi Biotec) in the presence of protein transport inhibitors Golgi-stop and Golgi-plug (BD Bioscience). Cells stimulated with medium containing DMSO only or with PMA (50 ng/ml)/ionomycin (750 ng/ml) were used as negative and positive control, respectively. After stimulation, cells were stained for live/dead viability dye and surface antigens: anti-CD3-PE-Cy7 (Biolegend), anti-CD4-FITC (Biolegend), and anti-CD8-PerCP (Biolegend), followed by fixation and permeabilization by using BD Cytofix/Cytoperm kit (BD Bioscience). Cells were then intracellularly stained with anti-IFN-γ-PE (eBioscience) for splenocytes or with anti-GZMB-Pacific Blue (Biolegend) for lung-tissue isolated cells. Samples were processed with FACS LSR-Fortessa (BD). Dead cells were excluded based on forward and side scatters and live/dead viability staining. Data were analyzed using FlowJo (TreeStar).

Cellular response in lung. Equivalent portions of lung tissues were harvested from infected and control mice, minced, and digested with 0.05% collagenase type IV (Thermo Fisher Scientific) in RPMI 1640 Medium for 30 minutes at 37° C. After digestion, lung single-cell suspensions were made by passing lung homogenates through 70 μm cell strainers. Red blood cells were removed by using Red Cell Lysis Buffer (Sigma-Aldrich). Leukocytes were stained with the Fixable Viability Dye (eFluor 506, Thermo Fisher Scientific) for live/dead cell staining, blocked with Fcγ-R blocker, and stained with fluorochrome-labeled antibodies (Abs). The following Abs purchased from Thermo Fisher Scientific and Biolegend (San Diego Calif.): PE-Cy7-anti-CD3F (145-2C11), Pacific Blue-anti-CD4 (GK1.5), APC-Cy7-anti-CD8a (53-6.7), and FITC-anti-CD19 (1D3).

Animal SARS-CoV2 challenge and PCR quantification of viral loads and inflammatory gene expression in the lung. Two weeks after booster vaccination (either i.m. or i.n.) as described above, all mice were intranasally challenged with a mouse-adapted SARS-CoV2 CMA4 strain (TCID50: 2×10⁴ pfu) developed by Dr. Pei-Yong Shi Laboratory. Viral challenge was conducted at the ABSL-3 facility at UTMB. Two days after challenge, all mice were euthanized and equivalent portions of lung tissues were collected for RNA extraction and viral load analysis. Total RNA was extracted from lung tissues using the TRIzol reagent according to the manufacturer's instructions. RNA concentration and purity were determined using the multi-mode reader (BioTek). To quantify SARS-CoV2 viral RNA and mouse inflammatory expression, cDNA was synthesized from RNA using the iScript Reverse Transcription Supermix for RT-qPCR (Bio-Rad). Expression of SARS-CoV2 (S, E, RdRp RNA) and mouse inflammatory genes (CCL2, CCL3, CCL7, CXCL10, TNF-α, and IL-1β) was quantified by qPCR using iTaq Universal SYBR Green Supermix (Bio-Rad) and the CFX Connect Real-Time PCR Detection System (Bio-Rad). Primers for individual genes were shown in the Table 1. PCR reactions (20 μl) contained 10 μM primers, 90 ng of cDNA, 10 μl iTaq universal SYBR Green supermix (2×) (Bio-Rad) and molecular grade water. PCR cycling conditions were: 95° C. for 3 minutes, 45 cycles of 95° C. for 5 seconds, and 60° C. for 30 seconds. For each PCR reaction, mouse GAPDH was also quantified for normalization.

Statistical Analysis. All statistical analyses were performed using Graph-Pad Prism 8.0. Statistical comparison between the mock and vaccine groups was performed using unpaired student's t test. The values were presented either as mean or mean±SD where appropriate. Two-tailed p values were denoted, and p values <0.05 were considered significant.

TABLE 1
Primer sequences for quantitative PCR (SEQ ID NOS: 6-25, respectively).

| Target | Primer Sequence | SEQ ID NO. |
|---|---|---|
| SARS-CoV2 S | F: CAGGACAAGAACACACAGGAA | SEQ ID NO: 6 |
| | R: CAGGCAGGATTTGGGAGAAA | SEQ ID NO: 7 |
| SARS-CoV2 E | F: GGAAGAGACAGGTACGTTAAATA | SEQ ID NO: 8 |
| | R: AGCAGTACGCACACAATCGAA | SEQ ID NO: 9 |
| SARS-COV2 RdRP | F: GTCATGTGTGGCGGTTCACT | SEQ ID NO: 10 |
| | R: CAACACTATTAGCATAAGCAGTTGT | SEQ ID NO: 11 |
| Mouse GAPDH | F: AGAACATCATCCCTGCATCC | SEQ ID NO: 12 |
| | R: CACATTGGGGGTAGGAACAC | SEQ ID NO: 13 |
| Mouse CCL2 | F: TTAAAAACCTGGATCGGAACCAA | SEQ ID NO: 14 |
| | R: GCATTAGCTTCAGATTTACGGGT | SEQ ID NO: 15 |
| Mouse CCL3 | F: GTGTAGAGCAGGGGCTTGAG | SEQ ID NO: 16 |
| | R: AGAGTCCTCGATGTGGCTA | SEQ ID NO: 17 |
| Mouse CCL7 | F: CCACATGCTGCTATGTCAAGA | SEQ ID NO: 18 |
| | R: ACACCGACTACTGGTGATCCT | SEQ ID NO: 19 |
| Mouse CXCL10 | F: CCAAGTGCTGCCGTCATTTTC | SEQ ID NO: 20 |
| | R: GGCTCGCAGGGATGATTTCAA | SEQ ID NO: 21 |
| Mouse TNF-α | F: CTTGTTGCCTCCTCTTTTGC | SEQ ID NO: 22 |
| | R: TGGTCACCAAATCAGCGTTA | SEQ ID NO: 23 |
| Mouse IL-6 | F: CTGCAAGAGACTTCCATCCAG | SEQ ID NO: 24 |
| | R: AGTGGTATAGACAGGTCTGTTGG | SEQ ID NO: 25 |

This application describes a new recombinant, multigenic SARS-CoV-2 vaccine candidate based on the MVA vector that expresses both the viral N and S proteins. The immunogenicity study showed that the vaccine is highly immunogenic, and i.m. immunization with the vaccine induces robust, systemic T-cell and binding antibody response specific to both antigens. Another interesting finding of this study is that, unlike i.m. immunization, i.n. delivery of the vaccine induces robust cellular immunity in the lung and confers immune control of SARS-CoV-2 and its associated lung inflammation in the infected animals. Therefore, this study demonstrates that vaccine-induced immune responses can induce control of SARS-CoV-2 in the absence of neutralizing activity, which can be used for the development of pan-coronavirus vaccines that include other antigenic proteins from SARS-CoV-2 and/or from existing coronaviruses (SARS, MVERS, etc.), their variants, or combinations thereof.

Current SARS-CoV-2 vaccines, including those clinically approved and under development, focus on the viral S protein and aim for inducing strong neutralizing antibodies (reviewed in [23-25]). Information on the role of cellular immunity in the context of SARS-CoV-2 vaccination is limited. In addition to the S protein, this vaccine approach also incorporates the viral N protein which contains extra critical T-cell epitopes [30]. This immunogenicity study in mice showed that the MVA-S+N vaccine induced T-cell responses and binding antibodies to both antigens. As expected, the vaccine did not induce neutralizing activity against SARS-CoV-2, which was confirmed by two independent live-virus neutralizing tests (FIG. 2D). This lack of neutralizing activity is in line with several previous reports that pre-fusion stabilized mutations in the S gene is critical for the generation of SARS-CoV-2 neutralizing antibody response [18, 48-50]. Thus, this design provided an opportunity to investigate whether or not the host immune responses induced by S and N proteins could confer protection against or immune control of SARS-CoV-2 independent of neutralizing activity. Indeed, these data show that intranasal immunization with the vaccine induces strong local cellular immunity in the lung (FIGS. 4A-4F), a response that is not effectively induced by intramuscular immunization (FIG. 8) but is associated with the viral control observed in the challenge model (FIGS. 5B-5C). Given the considerable variations in the S protein sequences across different coronaviruses and the constant mutations of the S protein, including the generation of SARS-CoV-2 spike variants with partial escape from vaccine-induced immunity [52, 53], these data demonstrate that simultaneously targeting S protein and another conserved antigen of the virus to induce neutralization-independent immune protection is possible and confer some cross protection.

Analysis of SARS-CoV-2-infected patients revealed that the virus induces broad and strong memory CD4+ and CD8+ T cells in the convalescent individuals [54]. Among the viral proteins, the S protein contains immunodominant T-cell epitopes and stimulates abundant S-specific T-cell response in infected individuals [34-36]. Information derived from earlier studies on SARS-CoV-1 indicated that S-specific memory T cells may participate in immune control of the virus [26]. However, the role of S-induced T cells in protection against SARS-CoV-2 remains unclear. Compared to the S protein, the SARS-CoV-2 N protein contains extra critical T-cell epitopes but is less mutable and more conserved across different human coronaviruses [37]. Evidence indicated that functional CD4+ and CD8+ T-cell response to the N protein could be long-lasting [38] and that this response correlates with the control of SARS-CoV-2 and is cross reactive to SARS-CoV [30, 38]. In this study, it is demonstrated that intramuscular delivery of the MVA-S+N vaccine induces robust, systemic N-specific CD4+ and CD8+ T-cell responses in the spleen (FIGS. 3A-3B) and that intranasal delivery of the vaccine elicits strong N-specific CD4 and CD8 T-cell responses in the lung, especially the granzyme B-expressing CD8 T cells with cytotoxic potential (FIGS. 4A-4F). In addition to N, the vaccine, delivered either via i.m. or i.n. immunization, also induced S-specific CD4 and CD8 T-cell responses at magnitudes comparable to the N-specific T-cell responses. The i.n. immunization with the vaccine of the present invention is able to induce immune control of SARS-CoV-2, the relative contribution of N- or S-specific immune responses to the observed viral control in this model remains to be determined. This information is important not only for understanding the host immunity to SARS-CoV-2 during infection, but also for developing pan-coronavirus vaccines.

Induction of protective mucosal immunity is critical for vaccine strategies against infectious agents that invade the body through mucosal surface [55, 56]. Compared to systemic vaccination (e.g., i.m.), mucosal delivery is generally considered more effective in eliciting mucosal immunity [55, 56]. Current SARS-CoV-2 vaccines, including those clinically approved, were largely administered via systemic route (i.m.) [5, 8, 9, 13, 57, 58]. Interestingly, i.m. immunization with these vaccines effectively elicited neutralizing antibodies in blood as well as in the respiratory system that conferred protection against SARS-CoV-2. In these studies, compared to the antibody response in the BAL, information on induction of vaccine-specific cellular immunity in the respiratory system and lung by i.m. route is limited. In this study, the data showed that while i.m. immunization with the MVA-S+N induces systemic antibody (FIGS. 2A-2E) and T-cell (FIGS. 3A-3B) responses as well as the detection of binding IgG in the BAL (FIG. 2F), it does not induce strong cellular response in the lung (FIG. 8). In contrast, i.n. immunization with the vaccine elicits strong cellular response in the lung, especially the antigen-specific, Granzyme B-expressing CD8 T cells (FIGS. 4A-4F). This T-cell response likely mediates the immune control of SARS-CoV-2 in the lung observed in the challenge model (FIGS. 5A, 5B). Another interesting finding is that i.m. and i.n. immunizations with the MVA-S+N induce comparable levels of SARS-CoV-2 specific binding IgG in the BAL, indicating that the antibodies in BAL are likely distributed from the peripheral circulation after vaccination [5]. The vaccine approach described herein (i.n. administration) effectively controlled viral loads (FIG. 5B) and alleviated virus-induced inflammation (FIG. 6) in the lung. The present invention can be used to determine if the vaccine induces rapid viral control in the upper respiratory tracts (e.g. nasal wash) to confer immunity to viral transmission. The present invention can also be used to determine longitudinal effects of the vaccine on SARS-CoV-2 viral loads in both upper (nasal swab/wash) and lower respiratory tracts (BAL), as well as on the virus-induced pathology and diseases (e.g. weight loss, lung inflammation, and/or pneumonia).

Durability of vaccine-induced immunity is another critical issue for SARS-CoV-2 and pan-coronavirus vaccine development [23]. Studies on patients indicate that infection by other human coronaviruses can induce immunological memory ranging from months to years; however, long-term data on SARS-CoV-2-induced immunity remain lacking [59]. Monitoring immune responses in SARS-CoV-2 mRNA vaccinated individuals indicated that the vaccine-induced antibody response, while declining slightly over time, remains detectable 3 months after booster vaccination [60]. In this study, a longitudinal analysis of MVA-S+N vaccinated mice (i.m.) showed that the N and S protein specific antibody response in sera remains readily detectable 11 weeks post booster vaccination (FIGS. 7A, 7B), indicating that the antibody response induced by the vaccine is likely to be durable. The present invention can be used to study the durability of vaccine-induced T-cell responses. Previous studies on host immunity to related coronaviruses (e.g., SARS-CoV-1 and MERS) showed that cellular immunity against these viruses could be maintained for longer periods of time compared to antibody responses [32, 33]. The present invention can be used to investigate the durability and long-term protection of immune response induced by the vaccine of the present invention via the intranasal route (FIGS. 4A-4F).

MVA as a viral vector has demonstrated favorable safety profiles [61] and has been used in vaccine development for a variety of infections, including SARS-CoV-2 [14, 17, 18]. Two recent studies reported that MVA encoding the pre-fusion stabilized S only induced neutralizing antibodies and host protection against SARS-CoV2 in animal models [14, 18]. Another study using synthetic MVA also indicated that multigenic MVA-SARS-CoV-2 vaccine candidate is immunogenic in mice, although it was not clear in that study if the vaccine was protective [17]. It was found herein that MVA as a vaccine vector is effective in expressing SARS-CoV-2 transgenes and effectively induces vaccine-specific immune responses in vivo. However, the present invention uses a distinct approach and provided evidence that mucosal delivery of MVA encoding both N and S proteins induced host protection primarily through cellular immunity in the lung.

In summary, this study demonstrates a new multigenic SARS-CoV2 immunization based on an MVA vector expressing both viral N and S proteins. This study demonstrated that intranasal delivery of the vaccine elicits strong cellular immunity in the lung that likely contributes to the control of SARS-CoV-2 and virus-induced inflammation in mice independent of neutralizing activity. This invention provides for host protective immunity to SARS-CoV-2 infection, which can be for SARS-CoV-2 and pan-coronavirus vaccines.

SARS-CoV-2 S Gene AA Sequence:
SEQ ID NO: 1
MFVFLVLLPLVSSQCVNLTTRTQLPPAYTNSFTRGVYYPDKVFRSSVLHSTQDLFLPFFSNVTWFHAIHVSGTNGTKRFDNPVLPF
NDGVYFASTEKSNIIRGWIFGTTLDSKTQSLLIVNNATNVVIKVCEFQFCNDPFLGVYYHKNNKSWMESEFRVYSSANNCTFEYVS
QPFLMDLEGKQGNFKNLREFVFKNIDGYFKIYSKHTPINLVRDLPQGFSALEPLVDLPIGINITRFQTLLALHRSYLTPGDSSSGW
TAGAAAYYVGYLQPRTFLLKYNENGTITDAVDCALDPLSETKCTLKSFTVEKGIYQTSNFRVQPTESIVRFPNITNLCPFGEVFNA
TRFASVYAWNRKRISNCVADYSVLYNSASFSTFKCYGVSPTKLNDLCFTNVYADSFVIRGDEVRQIAPGQTGKIADYNYKLPDDFT
GCVIAWNSNNLDSKVGGNYNYLYRLFRKSNLKPFERDISTEIYQAGSTPCNGVEGFNCYFPLQSYGFQPTNGVGYQPYRVVVLSFE
LLHAPATVCGPKKSTNLVKNKCVNFNFNGLTGTGVLTESNKKFLPFQQFGRDIADTTDAVRDPQTLEILDITPCSFGGVSVITPGT
NTSNQVAVLYQDVNCTEVPVAIHADQLTPTWRVYSTGSNVFQTRAGCLIGAEHVNNSYECDIPIGAGICASYQTQTNSPRRARSVA
SQSIIAYTMSLGAENSVAYSNNSIAIPTNFTISVTTEILPVSMTKTSVDCTMYICGDSTECSNLLLQYGSFCTQLNRALTGIAVEQ
DKNTQEVFAQVKQIYKTPPIKDFGGFNFSQILPDPSKPSKRSFIEDLLFNKVTLADAGFIKQYGDCLGDIAARDLICAQKFNGLTV
LPPLLTDEMIAQYTSALLAGTITSGWTFGAGAALQIPFAMQMAYRFNGIGVTQNVLYENQKLIANQFNSAIGKIQDSLSSTASALG
KLQDVVNQNAQALNTLVKQLSSNFGAISSVLNDILSRLDKVEAEVQIDRLITGRLQSLQTYVTQQLIRAAEIRASANLAATKMSEC
VLGQSKRVDFCGKGYHLMSFPQSAPHGVVFLHVTYVPAQEKNFTTAPAICHDGKAHFPREGVFVSNGTHWFVTQRNFYEPQIITTD
NTFVSGNCDVVIGIVNNTVYDPLQPELDSFKEELDKYFKNHTSPDVDLGDISGINASVVNIQKEIDRLNEVAKNLNESLIDLQELG
KYEQYIKWPWYIWLGFIAGLIAIVMVTIMLCCMTSCCSCLKGCCSCGSCCKFDEDDSEPVLKGVKLHYT*
SARS-CoV-2 S Gene nucleotide Sequence:
SEQ ID NO: 2
atgtttgtttttcttgttttattgccactagtctctagtcagtgtgttaatcttacaaccagaactcaattaccccctgcatacac
taattctttcacacgtggtgtttattaccctgacaaagttttcagatcctcagttttacattcaactcaggacttgttcttacctt
tcttttccaatgttacttggttccatgctatacatgtctctgggaccaatggtactaagaggtttgataaccctgtcctaccattt
aatgatggtgtttattttgcttccactgagaagtctaacataataagaggctggatttttggtactactttagattcgaagaccca
gtccctacttattgttaataacgctactaatgttgttattaaagtctgtgaatttcaattttgtaatgatccatttttgggtgttt
attaccacaaaaacaacaaaagttggatggaaagtgagttcagagtttattctagtgcgaataattgcacttttgaatatgtctct
cagccttttcttatggaccttgaaggaaaacagggtaatttcaaaaatcttagggaatttgtgtttaagaatattgatggttattt
taaaatatattctaagcacacgcctattaatttagtgcgtgatctccctcagggtttttcggctttagaaccattggtagatttgc
caataggtattaacatcactaggtttcaaactttacttgctttacatagaagttatttgactcctggtgattcttcttcaggttgg
acagctggtgctgcagcttattatgtgggttatcttcaacctaggacttttctattaaaatataatgaaaatggaaccattacaga
tgctgtagactgtgcacttgaccctctctcagaaacaaagtgtacgttgaaatccttcactgtagaaaaaggaatctatcaaactt
ctaactttagagtccaaccaacagaatctattgttagatttcctaatattacaaacttgtgcccttttggtgaagtttttaacgcc
accagatttgcatctgtttatgcttggaacaggaagagaatcagcaactgtgttgctgattattctgtcctatataattccgcatc
attttccacttttaagtgttatggagtgtctcctactaaattaaatgatctctgctttactaatgtctatgcagattcatttgtaa
ttagaggtgatgaagtcagacaaatcgctccagggcaaactggaaagattgctgattataattataaattaccagatgattttaca
ggctgcgttatagcttggaattctaacaatcttgattctaaggttggtggtaattataattacctgtatagattgtttaggaagtc
taatctcaaaccttttgagagagatatttcaactgaaatctatcaggccggtagcacaccttgtaatggtgttgaaggttttaatt
gttactttcctttacaatcatatggtttccaacccactaatggtgttggttaccaaccatacagagtagtagtactttcttttgaa
cttctacatgcaccagcaactgtttgtggacctaaaaagtctactaatttggttaaaaacaaatgtgtcaatttcaacttcaatgg
tttaacaggcacaggtgttcttactgagtctaacaaaaagtttctgcctttccaacaatttggcagagacattgctgacactactg
atgctgtccgtgatccacagacacttgagattcttgacattacaccatgttcttttggtggtgtcagtgttataacaccaggaaca
aatacttctaaccaggttgctgttctttatcaggatgttaactgcacagaagtccctgttgctattcatgcagatcaacttactcc
tacttggcgtgtttattctacaggttctaatgtttttcaaacacgtgcaggctgtttaataggggctgaacatgtcaacaactcat
atgagtgtgacatacccattggtgcaggtatatgcgctagttatcagactcagactaattctcctcggcgggcacgtagtgtagct
agtcaatccatcattgcctacactatgtcacttggtgcagaaaattcagttgcttactctaataactctattgccatacccacaaa
ttttactattagtgttaccacagaaattctaccagtgtctatgaccaagacatcagtagattgtacaatgtacatttgtggtgatt
caactgaatgcagcaatcttttgttgcaatatggcagtttttgtacacaattaaaccgtgctttaactggaatagctgttgaacaa
gacaaaaacacccaagaagtttttgcacaagtcaaacaaatttacaaaacaccaccaattaaagattttggtggttttaatttttc
acaaatattaccagatccatcaaaaccaagcaagaggtcatttattgaagatctacttttcaacaaagtgacacttgcagatgctg
gcttcatcaaacaatatggtgattgccttggtgatattgctgctagagacctcatttgtgcacaaaagtttaacggccttactgtt
ttgccacctttgctcacagatgaaatgattgctcaatacacttctgcactgttagcgggtacaatcacttctggttggacctttgg
tgcaggtgctgcattacaaataccatttgctatgcaaatggcttataggtttaatggtattggagttacacagaatgttctctatg
agaaccaaaaattgattgccaaccaatttaatagtgctattggcaaaattcaagactcactttcttccacagcaagtgcacttgga
aaacttcaagatgtggtcaaccaaaatgcacaagctttaaacacgcttgttaaacaacttagctccaattttggtgcaatttcaag
tgttttaaatgatatcctttcacgtcttgacaaagttgaggctgaagtgcaaattgataggttgatcacaggcagacttcaaagtt
tgcagacatatgtgactcaacaattaattagagctgcagaaatcagagcttctgctaatcttgctgctactaaaatgtcagagtgt
gtacttggacaatcaaaaagagttgatttttgtggaaagggctatcatcttatgtccttccctcagtcagcacctcatggtgtagt
cttcttgcatgtgacttatgtccctgcacaagaaaagaacttcacaactgctcctgccatttgtcatgatggaaaagcacactttc
ctcgtgaaggtgtctttgtttcaaatggcacacactggtttgtaacacaaaggaatttttatgaaccacaaatcattactacagac
aacacatttgtgtctggtaactgtgatgttgtaataggaattgtcaacaacacagtttatgatcctttgcaacctgaattagactc
attcaaggaggagttagataaatattttaagaatcatacatcaccagatgttgatttaggtgacatctctggcattaatgcttcag
ttgtaaacattcaaaaagaaattgaccgcctcaatgaggttgccaagaatttaaatgaatctctcatcgatctccaagaacttgga
aagtatgagcagtatataaaatggccatggtacatttggctaggttttatagctggcttgattgccatagtaatggtgacaattat
gctttgctgtatgaccagttgctgtagttgtctcaagggctgttgttcttgtggatcctgctgcaaatttgatgaagacgactctg
agccagtgctcaaaggagtcaaattacattacacaTAA
SARS-CoV-2 N Gene AA Sequence:
SEQ ID NO: 3
MSDNGPQNQRNAPRITFGGPSDSTGSNQNGERSGARSKQRRPQGLPNNTASWFTALTQHGKEDLKFPRGQGVPINTNSSPDDQIGY
YRRATRRIRGGDGKMKDLSPRWYFYYLGTGPEAGLPYGANKDGIIWVATEGALNTPKDHIGTRNPANNAAIVLQLPQGTTLPKGFY
AEGSRGGSQASSRSSSRSRNSSRNSTPGSSRGTSPARMAGNGGDAALALLLLDRLNQLESKMSGKGQQQQGQTVTKKSAAEASKKP
RQKRTATKAYNVTQAFGRRGPEQTQGNFGDQELIRQGTDYKHWPQIAQFAPSASAFFGMSRIGMEVTPSGTWLTYTGAIKLDDKDP
NFKDQVILLNKHIDAYKTFPPTEPKKDKKKKADETQALPQRQKKQQTVTLLPAADLDDFSKQLQQSMSSADSTQA*
SARS-CoV-2 N Gene nucleotide Sequence:
SEQ ID NO: 4
atgtctgataatggaccccaaaatcagcgaaatgcaccccgcattacgtttggtggaccctcagattcaactggcagtaaccagaa
tggagaacgcagtggggcgcgatcaaaacaacgtcggccccaaggtttacccaataatactgcgtcttggttcaccgctctcactc
aacatggcaaggaagaccttaaattccctcgaggacaaggcgttccaattaacaccaatagcagtccagatgaccaaattggctac
taccgaagagctaccagacgaattcgtggtggtgacggtaaaatgaaagatctcagtccaagatggtatttctactacctaggaac
tgggccagaagctggacttccctatggtgctaacaaagacggcatcatatgggttgcaactgagggagccttgaatacaccaaaag
atcacattggcacccgcaatcctgctaacaatgctgcaatcgtgctacaacttcctcaaggaacaacattgccaaaaggcttctac
gcagaagggagcagaggcggcagtcaagcctcttctcgttcctcatcacgtagtcgcaacagttcaagaaattcaactccaggcag
cagtaggggaacttctcctgctagaatggctggcaatggcggtgatgctgctcttgctttgctgctgcttgacagattgaaccagc
ttgagagcaaaatgtctggtaaaggccaacaacaacaaggccaaactgtcactaagaaatctgctgctgaggcttctaagaagcct
cggcaaaaacgtactgccactaaagcatacaatgtaacacaagctttcggcagacgtggtccagaacaaacccaaggaaattttgg
ggaccaggaactaatcagacaaggaactgattacaaacattggccgcaaattgcacaatttgcccccagcgcttcagcgttcttcg
gaatgtcgcgcattggcatggaagtcacaccttcgggaacgtggttgacctacacaggtgccatcaaattggatgacaaagatcca
aatttcaaagatcaagtcattttgctgaataagcatattgacgcatacaaaacattcccaccaacagagcctaaaaaggacaaaaa
gaagaaggctgatgaaactcaagccttaccgcagagacagaagaaacagcaaactgtgactcttcttcctgctgcagatttggatg
atttctccaaacaattgcaacaatccatgagcagtgctgactcaactcaggccTAA
Transfer plasmid:
SEQ ID NO: 5
CGGGGAGTCAGGCAACTATGGATGAACGAAATAGACAGATCGCTGAGATAGGTGCCTCACTGATTAAGCATTGGTAACTGTCAGAC
CAAGTTTACTCATATATACTTTAGATTGATTTAAAACTTCATTTTTAATTTAAAAGGATCTAGGTGAAGATCCTTTTTGATAATCT
CATGACCAAAATCCCTTAACGTGAGTTTTCGTTCCACTGAGCGTCAGACCCCGTAGAAAAGATCAAAGGATCTTCTTGAGATCCTT
TTTTTCTGCGCGTAATCTGCTGCTTGCAAACAAAAAAACCACCGCTACCAGCGGTGGTTTGTTTGCCGGATCAAGAGCTACCAACT
CTTTTTCCGAAGGTAACTGGCTTCAGCAGAGCGCAGATACCAAATACTGTCCTTCTAGTGTAGCCGTAGTTAGGCCACCACTTCAA
GAACTCTGTAGCACCGCCTACATACCTCGCTCTGCTAATCCTGTTACCAGTGGCTGCTGCCAGTGGCGATAAGTCGTGTCTTACCG
GGTTGGACTCAAGACGATAGTTACCGGATAAGGCGCAGCGGTCGGGCTGAACGGGGGGTTCGTGCACACAGCCCAGCTTGGAGCGA
ACGACCTACACCGAACTGAGATACCTACAGCGTGAGCTATGAGAAAGCGCCACGCTTCCCGAAGGGAGAAAGGCGGACAGGTATCC
GGTAAGCGGCAGGGTCGGAACAGGAGAGCGCACGAGGGAGCTTCCAGGGGGAAACGCCTGGTATCTTTATAGTCCTGTCGGGTTTC
GCCACCTCTGACTTGAGCGTCGATTTTTGTGATGCTCGTCAGGGGGGCGGAGCCTATCGAAAAACGCCAGCAACGCGGCCTTTTTA
CGGTTCCTGGCCTTTTGCTGGCCTTTTGCTCACATGTTCTTTCCTGCGTTATCCCCTGATTCTGTGGATAACCGTATTACCGCCTT
TGAGTGAGCTGATACCGCTCGCCGCAGCCGAACGACCGAGCGCAGCGAGTCAGTGAGCGAGGAAGCGGAAGAGCGCCCAATACGCA
AACCGCCTCTCCCCGCGCGTTGGCCGATTCATTAATGCAGCTGGCACGACAGGTTTCCCGACTGGAAAGCGGGCAGTGAGCGCAAC
GCAATTAATGTGAGTTAGCTCACTCATTAGGCACCCCAGGCTTTACACTTTATGCTTCCGGCTCGTATGTTGTGTGGAATTGTGAG
CGGATAACAATTTCACACAGGAAACAGCTATGACCATGATTACGCCAAGCTCTAATACGACTCACTATAGGGAGACAAGCTTAACT
AGTTTCTGGTGAATGTGTAGATCCAGATAGTATTATGTCTATAGTCGATTCACTATCTGTATTTACAATCAACTCTGTGGAGTATT
CTTCATAATCTACTTTATCAGTGTCATTTGTAGGCGATGTCATAAAGAATGCACATACATAAGTACCGGCATCTCTAGCAGTCAAT
GATTTAATTGTGATAGTTGTAACTAGATCATCGTATGGAGAGTCGTAAGATATTTTATCCTTGGTATAATTATCAAAATACAAGAC
GTCGCTTTTAGCAGCTAAAAGAATAATGGAATTGGGCTCCTTATACCAAGCACTCATAACAACGTAGTCATTTGTATTATTTCGAT
TACATGATAAAGTTGCATCATCACCTATTTTTTTAGATGTCTGAGGAAAAGGTGTAGCGTATACTAATGATATTAGTAACAAAAGT
ATTGGTAATCGTGTCATATTAGTATAAAAAGTGATTTATTTTTACAAAATTATGTATTTTGTTCTATCAACTACCTATAAAACTTT
CCCTGCAGCTAAAAATTGAAAATAAATACAAAGGTTCTTGAGGGTTGTGTTAAATTGAAAGCGAGAAATAATCATAAATAAGGTTG
ACTCTAGAGGATCCCCatgtctgataatggaccccaaaatcagcgaaatgcaccccgcattacgtttggtggaccctcagattcaa
ctggcagtaaccagaatggagaacgcagtggggcgcgatcaaaacaacgtcggccccaaggtttacccaataatactgcgtcttgg
ttcaccgctctcactcaacatggcaaggaagaccttaaattccctcgaggacaaggcgttccaattaacaccaatagcagtccaga
tgaccaaattggctactaccgaagagctaccagacgaattcgtggtggtgacggtaaaatgaaagatctcagtccaagatggtatt
tctactacctaggaactgggccagaagctggacttccctatggtgctaacaaagacggcatcatatgggttgcaactgagggagcc
ttgaatacaccaaaagatcacattggcacccgcaatcctgctaacaatgctgcaatcgtgctacaacttcctcaaggaacaacatt
gccaaaaggcttctacgcagaagggagcagaggcggcagtcaagcctcttctcgttcctcatcacgtagtcgcaacagttcaagaa
attcaactccaggcagcagtaggggaacttctcctgctagaatggctggcaatggcggtgatgctgctcttgctttgctgctgctt
gacagattgaaccagcttgagagcaaaatgtctggtaaaggccaacaacaacaaggccaaactgtcactaagaaatctgctgctga
ggcttctaagaagcctcggcaaaaacgtactgccactaaagcatacaatgtaacacaagctttcggcagacgtggtccagaacaaa
cccaaggaaattttggggaccaggaactaatcagacaaggaactgattacaaacattggccgcaaattgcacaatttgcccccagc
gcttcagcgttcttcggaatgtcgcgcattggcatggaagtcacaccttcgggaacgtggttgacctacacaggtgccatcaaatt
ggatgacaaagatccaaatttcaaagatcaagtcattttgctgaataagcatattgacgcatacaaaacattcccaccaacagagc
ctaaaaaggacaaaaagaagaaggctgatgaaactcaagccttaccgcagagacagaagaaacagcaaactgtgactcttcttcct
gctgcagatttggatgatttctccaaacaattgcaacaatccatgagcagtgctgactcaactcaggccggagcggccgccggaag
cggagctactaacttcagcctgctgaagcaggctggagacgtggaggagaaccctggacctATGGTGAGCAAAGGAGAGGCAGTGA
TAAAGGAATTCATGCGGTTTAAGGTCCACATGGAGGGATCCATGAACGGCCACGAATTTGAGATCGAAGGAGAAGGCGAGGGACGC
CCCTATGAGGGGACCCAGACCGCCAAGCTCAAGGTCACAAAGGGAGGCCCCCTGCCCTTCTCCTGGGATATCCTGTCCCCTCAGTT
TATGTACGGCTCCAGAGCCTTTACAAAGCACCCCGCCGATATACCAGACTACTACAAGCAGTCCTTCCCAGAAGGATTTAAGTGGG
AGCGCGTGATGAACTTTGAAGATGGCGGAGCCGTCACAGTGACCCAAGACACATCCCTGGAGGATGGCACCCTGATCTATAAAGTG
AAGCTCCGCGGCACCAACTTCCCTCCTGATGGACCCGTAATGCAGAAGAAGACAATGGGCTGGGAAGCGTCAACCGAGCGGTTGTA
CCCCGAGGACGGGGTGCTGAAGGGTGACATTAAAATGGCCCTGCGCCTGAAAGACGGAGGAAGGTACCTGGCTGACTTCAAAACCA
CATATAAGGCCAAGAAGCCCGTGCAGATGCCCGGCGCCTACAACGTCGACCGCAAGTTGGACATCACCTCCCATAACGAGGATTAC
ACCGTGGTGGAACAGTACGAACGCTCCGAAGGTCGCCACTCCACCGGCGGCATGGACGAGCTGTACAAGtaaGGGTACCCAGCCAC
CGAAAGAGCAATCTTAATCATGTCAGGTTCATATTTCCCCAACATACTAGATCCAAATTCGCCTACATCAGGTAAATTCATCATAA
TACAATGTCTGTTCATATCACACGATGATCCATTGAGCATCTCATCTCTATCAAGAATCCTAATCTGTGGTTCAAAATAACAGCAT
CTACACTCATCGTTAATTGTAGTATTGTCTAATATATTTTTGCTAATATTTGCGTAAGTTCTATTATCAGCTATTGCATGCATCAC
AGATCCATCAACAACCATATATAATATAGAACAATAGTCGGACTTTATACTTATGTAAAACTTGAACCAATTGGAACTCGGAAGCT
CGTCATGTAGACGCTGGTGTCTAGATATAATAACATTATTATCGGTTACACTTCTTAAGAGAGGTGCCGCATCGATAGAGAAATCA
AACAGGAGAATAATCAATGATGCATTTCCTTTGGTAAAAAAGGAAACATCCATGGGAAGAATGGCTACTTTATATGAATTTAACTC
TATACACGCACACGCTTTATCAGATGAGATTAATAGTTCACAAACATCTCTATCCTTTCCTATGGATATAATAACAGGAATGGCAT
CTTTAGGTTTAAAATAATTATATACACCAGTAGGAGTCTTGTCATCGTCATCTATCTTTATCAAATTAGCAAATCTGGATATTCTT
GATACATTCTTTTTATACAGTGAATTGCATACATCGGATACCGCATTATCCATATATGGCAAATCTGCAATCACTGTATTGTTTTT
AGATTGTCCGCCAATGTGAACGTTCTTGACTTTTTCACAACATGGTTTAATCATGAAATCATTTTTTATATGATTTATTTCCTCGC
CATGTTTTACTAACGCGTTTAGACAGTATACAATAACACCATCCATGGCGACCACCAAC

In one embodiment, the present invention includes an immunogenic protein for mucosal delivery comprising, consisting essentially of, or consisting of: at least 90% amino acid identity to a multigenic coronavirus vaccine on a modified vaccinia ankara (MVA) vector that expresses a viral nucleoprotein (N) protein and a spike (S) protein. In one aspect, the immunogenic protein is formulated into an intranasal, pulmonary, or alveolar immunization. In another aspect, the immunogenic protein are SEQ ID NOS:1 and 3. In another aspect, the immunogenic proteins are encoded by SEQ ID NOS: 2 and 4. In another aspect, an intranasal immunization induces at least one of: local T-cell responses in the lung; granzyme B-expressing cytotoxic CD8⁺ T cells; reduced viral loads; or reduced lung inflammation. In another aspect, the immunogenic protein is weakly or non-antigenic when administered intramuscularly. In another aspect, the immunogenic protein is effective without neutralizing antibodies. In another aspect, the immunogenic protein is included in a composition that further comprises an adjuvant selected from at least one of alum, aluminum hydroxide, aluminum phosphate, calcium phosphate hydroxide, cytosine-guanosine oligonucleotide (CpG-ODN) sequence, granulocyte macrophage colony stimulating factor (GM-CSF), monophosphoryl lipid A (MPL), poly(I:C), MF59, Quil A, N-acetyl muramyl-L-alanyl-D-isoglutamine (MDP), FIA, montanide, poly (DL-lactide-coglycolide), squalene, glucopyranosyl lipid adjuvant (GLA), GLA-Alum, 3M-052, a glucopyranosyl lipid adjuvant GLA emulsion with squalene (GLA-SE), virosome, AS03, ASO4, IL-1, IL-2, IL-3, IL-4, IL-5, IL-6, IL-7, IL-8, IL-10, IL-12, IL-15, IL-17, IL-18, STING, CD40L, pathogen-associated molecular patterns (PAMPs), damage-associated molecular pattern molecules (DAMPs), Freund's complete adjuvant, Freund's incomplete adjuvant, transforming growth factor (TGF)-beta antibody or antagonists, A2aR antagonists, lipopolysaccharides (LPS), Fas ligand, Trail, lymphotactin, Mannan (M-FP), APG-2, Hsp70 and Hsp90, pattern recognition receptor ligands, TLR3 ligands, TLR4 ligands, TLR5 ligands, TLR7/8 ligands, or TLR9 ligands. In another aspect, the coronavirus is MERS, SARS, SARS-CoV-2, or variants thereof.

In another embodiment, the present invention includes a method of stimulating an immune response in an animal comprising, consisting essentially of, or consisting of: administering to the animal a composition comprising at least 90% amino acid identity to a multigenic coronavirus vaccine on a modified vaccinia ankara (MVA) vector that expresses viral nucleoprotein (N) and S protein. In one aspect, the immunogenic proteins are formulated into an intranasal, pulmonary, or alveolar immunization. In another aspect, the antigenic proteins are SEQ ID NOS:1 and 3. In another aspect, the immunogenic proteins are encoded by SEQ ID NOS: 2 and 4. In another aspect, an intranasal immunization induces at least one of: local T-cell responses in the lung; granzyme B-expressing cytotoxic CD8⁺ T cells; reduced viral loads; or reduced lung inflammation. In another aspect, the immunogenic protein is weakly or non-antigenic when administered intramuscularly. In another aspect, the immunogenic protein is effective without neutralizing antibodies. In another aspect, the method further comprises adding an adjuvant selected from at least one of alum, aluminum hydroxide, aluminum phosphate, calcium phosphate hydroxide, cytosine-guanosine oligonucleotide (CpG-ODN) sequence, granulocyte macrophage colony stimulating factor (GM-CSF), monophosphoryl lipid A (MPL), poly(I:C), MF59, Quil A, N-acetyl muramyl-L-alanyl-D-isoglutamine (MDP), FIA, montanide, poly (DL-lactide-coglycolide), squalene, glucopyranosyl lipid adjuvant (GLA), GLA-Alum, 3M-052, a glucopyranosyl lipid adjuvant GLA emulsion with squalene (GLA-SE), virosome, AS03, ASO4, IL-1, IL-2, IL-3, IL-4, IL-5, IL-6, IL-7, IL-8, IL-10, IL-12, IL-15, IL-17, IL-18, STING, CD40L, pathogen-associated molecular patterns (PAMPs), damage-associated molecular pattern molecules (DAMPs), Freund's complete adjuvant, Freund's incomplete adjuvant, transforming growth factor (TGF)-beta antibody or antagonists, A2aR antagonists, lipopolysaccharides (LPS), Fas ligand, Trail, lymphotactin, Mannan (M-FP), APG-2, Hsp70 and Hsp90, pattern recognition receptor ligands, TLR3 ligands, TLR4 ligands, TLR5 ligands, TLR7/8 ligands, or TLR9 ligands. In another aspect, the coronavirus is MERS, SARS, SARS-CoV-2, or variants thereof.

In another embodiment, the present invention includes a method for production of an immunogenic protein comprising, consisting essentially of, or consisting of: (a) providing a cell containing an expression cassette having a nucleic acid encoding an immunogenic protein that has at least 90% amino acid identity to a multigenic SARS-CoV-2 vaccine on a modified vaccinia ankara (MVA) vector that expresses viral nucleoprotein (N) and S protein; and (b) growing the virus in a cell under conditions in which the nucleic acid is expressed and the immunogenic protein is produced. In one aspect, the method further comprises the step of recovering the immunogenic protein. In another aspect, a promoter is selected from the group consisting of constitutive promoters and tissue-specific promoters. In another aspect, the immunogenic proteins are formulated into an immunization. In another aspect, the immunogenic protein is formulated into an intranasal, pulmonary, or alveolar immunization. In another aspect, the antigenic proteins are SEQ ID NOS:1 and 3. In another aspect, the immunogenic proteins are encoded by SEQ ID NOS: 2 and 4. In another aspect, the intranasal immunization induces at least one of: local T-cell responses in the lung; granzyme B-expressing cytotoxic CD8⁺ T cells; reduced viral loads; or reduced lung inflammation. In another aspect, the immunogenic protein is weakly or non-antigenic when administered intramuscularly. In another aspect, the immunogenic protein is effective without neutralizing antibodies. In another aspect, the coronavirus is MERS, SARS, SARS-CoV-2, or variants thereof.

In another embodiment, the present invention includes a nucleic acid encoding a protein comprising, consisting essentially of, or consisting of: an immunogenic protein that has at least 90% amino acid identity to a multigenic coronavirus vaccine on a modified vaccinia ankara (MVA) vector that expresses viral nucleoprotein (N) and S protein. In one aspect, the antigenic proteins are SEQ ID NOS:1 and 3. In another aspect, the immunogenic proteins are encoded by SEQ ID NOS: 2 and 4. In another aspect, the coronavirus is MERS, SARS, or SARS-CoV-2, or variants thereof.

In another embodiment, the present invention includes a vector that comprises, consisting essentially of, or consisting of: a nucleic acid that encodes an immunogenic protein that has at least 90% amino acid identity to a multigenic coronavirus vaccine on a modified vaccinia ankara (MVA) vector that expresses a viral nucleoprotein (N) protein and a spike (S) protein.

In another embodiment, the present invention includes a host cell that comprises, consisting essentially of, or consisting of: a vector that expresses an immunogenic protein that has at least 90% amino acid identity to a multigenic coronavirus vaccine on a modified vaccinia ankara (MVA) vector that expresses a viral nucleoprotein (N) protein and a spike (S) protein.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

1. Zhu, N., et al., A Novel Coronavirus from Patients with Pneumonia in China, 2019. N Engl J Med, 2020. 382(8): p. 727-733.
2. Amanat, F. and F. Krammer, SARS-CoV-2 Vaccines: Status Report. Immunity, 2020. 52(4): p. 583-589.
3. Funk, C. D., C. Laferrière, and A. Ardakani, A Snapshot of the Global Race for Vaccines Targeting SARS-CoV-2 and the COVID-19 Pandemic. Frontiers in Pharmacology, 2020. 11(937).
4. Graham, B. S., Rapid COVID-19 vaccine development. Science, 2020. 368(6494): p. 945-946.
5. Smith, T. R. F., et al., Immunogenicity of a DNA vaccine candidate for COVID-19. Nat Commun, 2020. 11(1): p. 2601.
6. Yu, J., et al., DNA vaccine protection against SARS-CoV-2 in rhesus macaques. Science, 2020.
7. Corbett, K. S., et al., Evaluation of the mRNA-1273 Vaccine against SARS-CoV-2 in Nonhuman Primates. N Engl J Med, 2020.
8. Polack, F. P., et al., Safety and Efficacy of the BNT162b2 mRNA Covid-19 Vaccine. N Engl J Med, 2020. 383(27): p. 2603-2615.
9. Baden, L. R., et al., Efficacy and Safety of the mRNA-1273 SARS-CoV-2 Vaccine. N Engl J Med, 2021. 384(5): p. 403-416.
10. Case, J. B., et al., Replication-Competent Vesicular Stomatitis Virus Vaccine Vector Protects against SARS-CoV-2-Mediated Pathogenesis in Mice. Cell Host Microbe, 2020. 28(3): p. 465-474 e4.
11. Zhu, F. C., et al., Safety, tolerability, and immunogenicity of a recombinant adenovirus type-5 vectored COVID-19 vaccine: a dose-escalation, open-label, non-randomised, first-in-human trial. Lancet, 2020. 395(10240): p. 1845-1854.
12. van Doremalen, N., et al., ChAdOx1 nCoV-19 vaccine prevents SARS-CoV-2 pneumonia in rhesus macaques. Nature, 2020.
13. Mercado, N. B., et al., Single-shot Ad26 vaccine protects against SARS-CoV-2 in rhesus macaques. Nature, 2020.
14. Routhu, N. K., et al., A modified vaccinia Ankara vector-based vaccine protects macaques from SARS-CoV-2 infection, immune pathology, and dysfunction in the lungs. Immunity, 2021.
15. Hassan, A. O., et al., A Single-Dose Intranasal ChAd Vaccine Protects Upper and Lower Respiratory Tracts against SARS-CoV-2. Cell, 2020.
16. Lu, M., et al., A safe and highly efficacious measles virus-based vaccine expressing SARS-CoV-2 stabilized prefusion spike. Proc Natl Acad Sci USA, 2021. 118(12).
17. Chiuppesi, F., et al., Development of a multi-antigenic SARS-CoV-2 vaccine candidate using a synthetic poxvirus platform. Nat Commun, 2020. 11(1): p. 6121.
18. Liu, R., et al., MVA Vector Vaccines Inhibit SARS CoV-2 Replication in Upper and Lower Respiratory Tracts of Transgenic Mice and Prevent Lethal Disease. bioRxiv, 2021.
19. Yang, J., et al., A vaccine targeting the RBD of the S protein of SARS-CoV-2 induces protective immunity. Nature, 2020.
20. Dai, L., et al., A Universal Design of Betacoronavirus Vaccines against COVID-19, MERS, and SARS. Cell, 2020.
21. Keech, C., et al., Phase 1-2 Trial of a SARS-CoV-2 Recombinant Spike Protein Nanoparticle Vaccine. N Engl J Med, 2020. 383(24): p. 2320-2332.
22. Gao, Q., et al., Development of an inactivated vaccine candidate for SARS-CoV-2. Science, 2020.
23. Kyriakidis, N. C., et al., SARS-CoV-2 vaccines strategies: a comprehensive review of phase 3 candidates. NPJ Vaccines, 2021. 6(1): p. 28.
24. Poland, G. A., I. G. Ovsyannikova, and R. B. Kennedy, SARS-CoV-2 immunity: review and applications to phase 3 vaccine candidates. Lancet, 2020. 396(10262): p. 1595-1606.
25. Krammer, F., SARS-CoV-2 vaccines in development. Nature, 2020. 586(7830): p. 516-527.
26. Channappanavar, R., et al., Virus-specific memory CD8 T cells provide substantial protection from lethal severe acute respiratory syndrome coronavirus infection. J Virol, 2014. 88(19): p. 11034-44.
27. Le Bert, N., et al., SARS-CoV-2-specific T cell immunity in cases of COVID-19 and SARS, and uninfected controls. Nature, 2020.
28. Grifoni, A., et al., Targets of T Cell Responses to SARS-CoV-2 Coronavirus in Humans with COVID-19 Disease and Unexposed Individuals. Cell, 2020. 181(7): p. 1489-1501 e15.
29. Sekine, T., et al., Robust T Cell Immunity in Convalescent Individuals with Asymptomatic or Mild COVID-19. Cell, 2020. 183(1): p. 158-168 e14.
30. Peng, Y., et al., Broad and strong memory CD4(+) and CD8(+) T cells induced by SARS-CoV-2 in UK convalescent individuals following COVID-19. Nat Immunol, 2020.
31. Wu, L. P., et al., Duration of antibody responses after severe acute respiratory syndrome. Emerg Infect Dis, 2007. 13(10): p. 1562-4.
32. Yang, L. T., et al., Long-lived effector/central memory T-cell responses to severe acute respiratory syndrome coronavirus (SARS-CoV) S antigen in recovered SARS patients. Clin Immunol, 2006. 120(2): p. 171-8.
33. Peng, H., et al., Long-lived memory T lymphocyte responses against SARS coronavirus nucleocapsid protein in SARS-recovered patients. Virology, 2006. 351(2): p. 466-75.
34. Altmann, D. M. and R. J. Boyton, SARS-CoV-2 T cell immunity: Specificity, function, durability, and role in protection. Sci Immunol, 2020. 5(49).
35. Sattler, A., et al., SARS-CoV-2-specific T cell responses and correlations with COVID-19 patient predisposition. J Clin Invest, 2020. 130(12): p. 6477-6489.
36. de Candia, P., et al., T Cells: Warriors of SARS-CoV-2 Infection. Trends Immunol, 2021. 42(1): p. 18-30.
37. Grifoni, A., et al., A Sequence Homology and Bioinformatic Approach Can Predict Candidate Targets for Immune Responses to SARS-CoV-2. Cell Host Microbe, 2020. 27(4): p. 671-680 e2.
38. Le Bert, N., et al., SARS-CoV-2-specific T cell immunity in cases of COVID-19 and SARS, and uninfected controls. Nature, 2020. 584(7821): p. 457-462.
39. Dutta, N. K., K. Mazumdar, and J. T. Gordy, The Nucleocapsid Protein of SARS-CoV-2: a Target for Vaccine Development. J Virol, 2020. 94(13).
40. Wyatt, L. S., P. L. Earl, and B. Moss, Generation of Recombinant Vaccinia Viruses. Curr Protoc Protein Sci, 2017. 89: p. 5 13 1-5 13 18.
41. Earl, P. L., et al., Design and evaluation of multi-gene, multi-clade HIV-1 MVA vaccines. Vaccine, 2009. 27(42): p. 5885-95.
42. Harcourt, J., et al., Isolation and characterization of SARS-CoV-2 from the first US COVID-19 patient. bioRxiv, 2020.
43. Baker, C. and P. Y. Shi, Construction of Stable Reporter Flaviviruses and Their Applications. Viruses, 2020. 12(10).
44. Garcia-Arriaza, J., et al., Immunogenic profiling in mice of a HIV/AIDS vaccine candidate (MVA-B) expressing four HIV-1 antigens and potentiation by specific gene deletions. PLoS One, 2010. 5(8): p. e12395.
45. Routhu, N. K., et al., Modified Vaccinia Ankara Based SARS-CoV-2 Vaccine Expressing Full-Length Spike Induces Strong Neutralizing Antibody Response. bioRxiv, 2020: p. 2020.06.27.175166.
46. Okba, N. M. A., et al., Severe Acute Respiratory Syndrome Coronavirus 2-Specific Antibody Responses in Coronavirus Disease Patients. Emerg Infect Dis, 2020. 26(7): p. 1478-1488.
47. Muruato, A. E., et al., A high-throughput neutralizing antibody assay for COVID-19 diagnosis and vaccine evaluation. Nat Commun, 2020. 11(1): p. 4059.
48. Pallesen, J., et al., Immunogenicity and structures of a rationally designed prefusion MERS-CoV spike antigen. Proc Natl Acad Sci USA, 2017. 114(35): p. E7348-E7357.
49. Cai, Y., et al., Distinct conformational states of SARS-CoV-2 spike protein. Science, 2020. 369(6511): p. 1586-1592.
50. Bos, R., et al., Ad26 vector-based COVID-19 vaccine encoding a prefusion-stabilized SARS-CoV-2 Spike immunogen induces potent humoral and cellular immune responses. NPJ Vaccines, 2020. 5: p. 91.
51. Harrison, A. G., T. Lin, and P. Wang, Mechanisms of SARS-CoV-2 Transmission and Pathogenesis. Trends Immunol, 2020. 41(12): p. 1100-1115.
52. Liu, Y., et al., Neutralizing Activity of BNT162b2-Elicited Serum—Preliminary Report. N Engl J Med, 2021.
53. Xie, X., et al., Neutralization of SARS-CoV-2 spike 69/70 deletion, E484K and N501Y variants by BNT162b2 vaccine-elicited sera. Nat Med, 2021.
54. Peng, Y., et al., Broad and strong memory CD4(+) and CD8(+) T cells induced by SARS-CoV-2 in UK convalescent individuals following COVID-19. Nat Immunol, 2020. 21(11): p. 1336-1345.
55. Su, F., et al., Induction of mucosal immunity through systemic immunization: Phantom or reality? Hum Vaccin Immunother, 2016. 12(4): p. 1070-9.
56. Neutra, M. R. and P. A. Kozlowski, Mucosal vaccines: the promise and the challenge. Nat Rev Immunol, 2006. 6(2): p. 148-58.
57. Tostanoski, L. H., et al., Ad26 vaccine protects against SARS-CoV-2 severe clinical disease in hamsters. Nat Med, 2020. 26(11): p. 1694-1700.
58. Routhu, N. K., et al., A modified vaccinia Ankara vector-based vaccine protects macaques from SARS-CoV-2 infection, immune pathology, and dysfunction in the lungs. Immunity, 2021. 54(3): p. 542-556 e9.
59. Dan, J. M., et al., Immunological memory to SARS-CoV-2 assessed for up to 8 months after infection. Science, 2021. 371(6529).
60. Widge, A. T., et al., Durability of Responses after SARS-CoV-2 mRNA-1273 Vaccination. N Engl J Med, 2021. 384(1): p. 80-82.
61. Prow, N. A., et al., Poxvirus-based vector systems and the potential for multi-valent and multi-pathogen vaccines. Expert Rev Vaccines, 2018. 17(10): p. 925-934.
62. Xie, X., et al., An Infectious cDNA Clone of SARS-CoV-2. Cell Host Microbe, 2020. 27(5): p. 841-848 e3.
63. Wyatt, L. S., P. L. Earl, and B. Moss, Generation of Recombinant Vaccinia Viruses. Curr Protoc Mol Biol, 2017. 117: p. 16 17 1-16 17 18.
64. Choi, E. J., et al., The role of M2-2 PDZ-binding motifs in pulmonary innate immune responses to human metapneumovirus. J Med Virol, 2020.

What is claimed is:

1. An immunogenic protein for mucosal delivery comprising:
at least 90%, 91%, 92%, 93%, 94%, 95%, 96%, 97%, 98%, 99%, or 100% amino acid identity to a multigenic coronavirus vaccine on a modified vaccinia ankara (MVA) vector that expresses a viral nucleoprotein (N) protein and a spike (S) protein.

2. The immunogenic protein of claim 1, wherein the immunogenic protein is formulated into an intranasal, pulmonary, or alveolar immunization.

3. The immunogenic protein of claim 1, wherein the amino acid sequence is SEQ ID NO:1, 3, or both.

4. The immunogenic protein of claim 1, wherein the immunogenic protein is encoded by the nucleic acid of SEQ ID NO: 2, 4, or both.

5. The immunogenic protein of claim 1, wherein the mucosal delivery is an intranasal immunization that induces at least one of: local T-cell responses in the lung; granzyme B-expressing cytotoxic CD8⁺ T cells; reduced viral loads; or reduced lung inflammation; the immunogenic protein is weakly or non-antigenic when administered intramuscularly; or the immunogenic protein is effective without neutralizing antibodies.

6. The immunogenic protein of claim 1, further comprising an adjuvant selected from at least one of alum, aluminum hydroxide, aluminum phosphate, calcium phosphate hydroxide, cytosine-guanosine oligonucleotide (CpG-ODN) sequence, granulocyte macrophage colony stimulating factor (GM-CSF), monophosphoryl lipid A (MPL), poly(I:C), MF59, Quil A, N-acetyl muramyl-L-alanyl-D-isoglutamine (MDP), FIA, montanide, poly (DL-lactide-coglycolide), squalene, glucopyranosyl lipid adjuvant (GLA), GLA-Alum, 3M-052, a glucopyranosyl lipid adjuvant GLA emulsion with squalene (GLA-SE), virosome, AS03, ASO4, IL-1, IL-2, IL-3, IL-4, IL-5, IL-6, IL-7, IL-8, IL-10, IL-12, IL-15, IL-17, IL-18, STING, CD40L, pathogen-associated molecular patterns (PAMPs), damage-associated molecular pattern molecules (DAMPs), Freund's complete adjuvant, Freund's incomplete adjuvant, transforming growth factor (TGF)-beta antibody or antagonists, A2aR antagonists, lipopolysaccharides (LPS), Fas ligand, Trail, lymphotactin, Mannan (M-FP), APG-2, Hsp70 and Hsp90, pattern recognition receptor ligands, TLR3 ligands, TLR4 ligands, TLR5 ligands, TLR7/8 ligands, or TLR9 ligands.

7. The immunogenic protein of claim 1, wherein the coronavirus is MERS, SARS, SARS-CoV-2, or variants thereof.

8. A method of stimulating an immune response in an animal comprising administering to the animal a composition comprising an immunogenic protein with at least 90%, 91%, 92%, 93%, 94%, 95%, 96%, 97%, 98%, 99%, or 100% amino acid identity to a multigenic coronavirus vaccine on a modified vaccinia ankara (MVA) vector that expresses a viral nucleoprotein (N) and a Spike (S) protein.

9. The method of claim 8, wherein the immunogenic protein is formulated into an intranasal, pulmonary, or alveolar immunization.

10. The method of claim 8, wherein the amino acid sequence is SEQ ID NO:1, 3, or both.

11. The method of claim 8, wherein the immunogenic protein is encoded by the nucleic acid of SEQ ID NO: 2, 4, or both.

12. The method of claim 8, wherein the administration is by an intranasal immunization that induces at least one of: local T-cell responses in the lung; granzyme B-expressing cytotoxic CD8⁺ T cells; reduced viral loads; or reduced lung inflammation; the immunogenic protein is weakly or non-antigenic when administered intramuscularly; the immunogenic protein is effective without neutralizing antibodies; or the coronavirus is MERS, SARS, SARS-CoV-2, or variants thereof.

13. The method of claim 8, further comprising an adjuvant selected from at least one of alum, aluminum hydroxide, aluminum phosphate, calcium phosphate hydroxide, cytosine-guanosine oligonucleotide (CpG-ODN) sequence, granulocyte macrophage colony stimulating factor (GM-CSF), monophosphoryl lipid A (MPL), poly(I:C), MF59, Quil A, N-acetyl muramyl-L-alanyl-D-isoglutamine (MDP), FIA, montanide, poly (DL-lactide-coglycolide), squalene, glucopyranosyl lipid adjuvant (GLA), GLA-Alum, 3M-052, a glucopyranosyl lipid adjuvant GLA emulsion with squalene (GLA-SE), virosome, AS03, ASO4, IL-1, IL-2, IL-3, IL-4, IL-5, IL-6, IL-7, IL-8, IL-10, IL-12, IL-15, IL-17, IL-18, STING, CD40L, pathogen-associated molecular patterns (PAMPs), damage-associated molecular pattern molecules (DAMPs), Freund's complete adjuvant, Freund's incomplete adjuvant, transforming growth factor (TGF)-beta antibody or antagonists, A2aR antagonists, lipopolysaccharides (LPS), Fas ligand, Trail, lymphotactin, Mannan (M-FP), APG-2, Hsp70 and Hsp90, pattern recognition receptor ligands, TLR3 ligands, TLR4 ligands, TLR5 ligands, TLR7/8 ligands, or TLR9 ligands.

14. A method for production of an immunogenic protein comprising:
(a) providing a cell containing an expression cassette having a nucleic acid encoding an immunogenic protein that has at least 90%, 91%, 92%, 93%, 94%, 95%, 96%, 97%, 98%, 99%, or 100% amino acid identity to a multigenic SARS-CoV-2 coronavirus vaccine on a modified vaccinia ankara (MVA) vector that expresses viral nucleoprotein (N) and Spike (S) protein; and
(b) growing the virus in a cell under conditions in which the nucleic acid is expressed and the immunogenic protein is produced.

15. The method of claim 14, further comprising the step of recovering the immunogenic protein.

16. The method of claim 14, wherein a promoter is selected from the group consisting of constitutive promoters and tissue specific promoters.

17. The method of claim 14, wherein the immunogenic protein is formulated into an immunization; or an intranasal, pulmonary, or alveolar immunization.

18. The method of claim 14, wherein the amino acid sequence is SEQ ID NO:1, 3, or both.

19. The method of claim 14, wherein the immunogenic protein is encoded by the nucleic acid of SEQ ID NO: 2, 4, or both.

20. The method of claim 14, wherein the intranasal immunization induces at least one of: local T-cell responses in the lung; granzyme B-expressing cytotoxic CD8⁺ T cells; reduced viral loads; or reduced lung inflammation; weakly or non-antigenic when administered intramuscularly; the immunogenic protein is effective without neutralizing antibodies; or the coronavirus is MERS, SARS, SARS-CoV-2, or variants thereof.

21. A nucleic acid encoding a protein comprising:
an immunogenic protein that has at least 90%, 91%, 92%, 93%, 94%, 95%, 96%, 97%, 98%, 99%, or 100% amino acid identity to a multigenic coronavirus vaccine on a modified vaccinia ankara (MVA) vector that expresses viral nucleoprotein (N) and S protein.

22. The nucleic acid of claim 21, wherein the wherein the amino acid sequence is SEQ ID NO:1, 3, or both.

23. The nucleic acid of claim 21, wherein the immunogenic protein is encoded by the nucleic acid of SEQ ID NO: 2, 4, or both.

24. The nucleic acid of claim 21, wherein the coronavirus is MERS, SARS, or SARS-CoV-2, or variants thereof.

25. A vector that comprises a nucleic acid that encodes an immunogenic protein that has at least 90%, 91%, 92%, 93%, 94%, 95%, 96%, 97%, 98%, 99%, or 100% amino acid identity to a multigenic coronavirus vaccine on a modified vaccinia ankara (MVA) vector that expresses a viral nucleoprotein (N) protein and a spike (S) protein.

26. A host cell that comprises a vector that expresses an immunogenic protein that has at least 90%, 91%, 92%, 93%, 94%, 95%, 96%, 97%, 98%, 99%, or 100% amino acid identity to a multigenic coronavirus vaccine on a modified vaccinia ankara (MVA) vector that expresses a viral nucleoprotein (N) protein and a spike (S) protein.


SEQUENCE LISTING (25 sequences)

SEQ ID NO: 1 - protein, 1273 aa, SARS-CoV-2
  misc_feature (1)..(1273): SARS-CoV-2 S Gene AA Sequence
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - DNA, 3822 bp, SARS-CoV-2
  misc_feature (1)..(3822): SARS-CoV-2 S Gene nucleotide Sequence
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 3 - protein, 419 aa, SARS-CoV-2
  misc_feature (1)..(419): SARS-CoV-2 N Gene AA Sequence
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 4 - DNA, 1260 bp, SARS-CoV-2
  misc_feature (1)..(1260): SARS-CoV-2 N Gene nucleotide Sequence
atgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaa

SEQ ID NO: 5 - DNA, 4875 bp, Artificial Sequence
  Synthetic Transfer plasmid
cggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatcgaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctaatacgactcactatagggagacaagcttaactagtttctggtgaatgtgtagatccagatagtattatgtctatagtcgattcactatctgtatttacaatcaactctgtggagtattcttcataatctactttatcagtgtcatttgtaggcgatgtcataaagaatgcacatacataagtaccggcatctctagcagtcaatgatttaattgtgatagttgtaactagatcatcgtatggagagtcgtaagatattttatccttggtataattatcaaaatacaagacgtcgcttttagcagctaaaagaataatggaattgggctccttataccaagcactcataacaacgtagtcatttgtattatttcgattacatgataaagttgcatcatcacctatttttttagatgtctgaggaaaaggtgtagcgtatactaatgatattagtaacaaaagtattggtaatcgtgtcatattagtataaaaagtgatttatttttacaaaattatgtattttgttctatcaactacctataaaactttccctgcagctaaaaattgaaaataaatacaaaggttcttgagggttgtgttaaattgaaagcgagaaataatcataaataaggttgactctagaggatccccatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggccggagcggccgccggaagcggagctactaacttcagcctgctgaagcaggctggagacgtggaggagaaccctggacctatggtgagcaaaggagaggcagtgataaaggaattcatgcggtttaaggtccacatggagggatccatgaacggccacgaatttgagatcgaaggagaaggcgagggacgcccctatgaggggacccagaccgccaagctcaaggtcacaaagggaggccccctgcccttctcctgggatatcctgtcccctcagtttatgtacggctccagagcctttacaaagcaccccgccgatataccagactactacaagcagtccttcccagaaggatttaagtgggagcgcgtgatgaactttgaagatggcggagccgtcacagtgacccaagacacatccctggaggatggcaccctgatctataaagtgaagctccgcggcaccaacttccctcctgatggacccgtaatgcagaagaagacaatgggctgggaagcgtcaaccgagcggttgtaccccgaggacggggtgctgaagggtgacattaaaatggccctgcgcctgaaagacggaggaaggtacctggctgacttcaaaaccacatataaggccaagaagcccgtgcagatgcccggcgcctacaacgtcgaccgcaagttggacatcacctcccataacgaggattacaccgtggtggaacagtacgaacgctccgaaggtcgccactccaccggcggcatggacgagctgtacaagtaagggtacccagccaccgaaagagcaatcttaatcatgtcaggttcatatttccccaacatactagatccaaattcgcctacatcaggtaaattcatcataatacaatgtctgttcatatcacacgatgatccattgagcatctcatctctatcaagaatcctaatctgtggttcaaaataacagcatctacactcatcgttaattgtagtattgtctaatatatttttgctaatatttgcgtaagttctattatcagctattgcatgcatcacagatccatcaacaaccatatataatatagaacaatagtcggactttatacttatgtaaaacttgaaccaattggaactcggaagctcgtcatgtagacgctggtgtctagatataataacattattatcggttacacttcttaagagaggtgccgcatcgatagagaaatcaaacaggagaataatcaatgatgcatttcctttggtaaaaaaggaaacatccatgggaagaatggctactttatatgaatttaactctatacacgcacacgctttatcagatgagattaatagttcacaaacatctctatcctttcctatggatataataacaggaatggcatctttaggtttaaaataattatatacaccagtaggagtcttgtcatcgtcatctatctttatcaaattagcaaatctggatattcttgatacattctttttatacagtgaattgcatacatcggataccgcattatccatatatggcaaatctgcaatcactgtattgtttttagattgtccgccaatgtgaacgttcttgactttttcacaacatggtttaatcatgaaatcattttttatatgatttatttcctcgccatgttttactaacgcgtttagacagtatacaataacaccatccatggcgaccaccaac

SEQ ID NO: 6 - DNA, 21 bp, Artificial Sequence
  Synthetic Primer target - SARS-CoV2 S
caggacaagaacacacaggaa

SEQ ID NO: 7 - DNA, 20 bp, Artificial Sequence
  Synthetic Primer target - SARS-CoV2 S
caggcaggatttgggagaaa

SEQ ID NO: 8 - DNA, 22 bp, Artificial Sequence
  Synthetic Primer target - SARS-CoV2 E
ggaagagacaggtacgttaata

SEQ ID NO: 9 - DNA, 21 bp, Artificial Sequence
  Synthetic Primer target - SARS-CoV2 E
agcagtacgcacacaatcgaa

SEQ ID NO: 10 - DNA, 20 bp, Artificial Sequence
  Synthetic Primer target - SARS-CoV2 RdRP
gtcatgtgtggcggttcact

SEQ ID NO: 11 - DNA, 25 bp, Artificial Sequence
  Synthetic Primer target - SARS-CoV2 RdRP
caacactattagcataagcagttgt

SEQ ID NO: 12 - DNA, 20 bp, Artificial Sequence
  Synthetic Primer target - Mouse GAPDH
agaacatcatccctgcatcc

SEQ ID NO: 13 - DNA, 20 bp, Artificial Sequence
  Synthetic Primer target - Mouse GAPDH
cacattgggggtaggaacac

SEQ ID NO: 14 - DNA, 23 bp, Artificial Sequence
  Synthetic Primer target - Mouse CCL2
ttaaaaacctggatcggaaccaa

SEQ ID NO: 15 - DNA, 23 bp, Artificial Sequence
  Synthetic Primer target - Mouse CCL2
gcattagcttcagatttacgggt

SEQ ID NO: 16 - DNA, 20 bp, Artificial Sequence
  Synthetic Primer target - Mouse CCL3
gtgtagagcaggggcttgag

SEQ ID NO: 17 - DNA, 19 bp, Artificial Sequence
  Synthetic Primer target - Mouse CCL3
agagtcctcgatgtggcta

SEQ ID NO: 18 - DNA, 21 bp, Artificial Sequence
  Synthetic Primer target - Mouse CCL7
ccacatgctgctatgtcaaga

SEQ ID NO: 19 - DNA, 21 bp, Artificial Sequence
  Synthetic Primer target - Mouse CCL7
acaccgactactggtgatcct

SEQ ID NO: 20 - DNA, 21 bp, Artificial Sequence
  Synthetic Primer target - Mouse CXCL10
ccaagtgctgccgtcattttc

SEQ ID NO: 21 - DNA, 21 bp, Artificial Sequence
  Synthetic Primer target - Mouse CXCL10
ggctcgcagggatgatttcaa

SEQ ID NO: 22 - DNA, 20 bp, Artificial Sequence
  Synthetic Primer target - Mouse TNF-a
cttgttgcctcctcttttgc

SEQ ID NO: 23 - DNA, 20 bp, Artificial Sequence
  Synthetic Primer target - Mouse TNF-a
tggtcaccaaatcagcgtta

SEQ ID NO: 24 - DNA, 21 bp, Artificial Sequence
  Synthetic Primer target - Mouse IL-6
ctgcaagagacttccatccag

SEQ ID NO: 25 - DNA, 23 bp, Artificial Sequence
  Synthetic Primer target - Mouse IL-6
agtggtatagacaggtctgttgg